(19) United States
(12) Patent Application Publication
(10) Pub. No.: US 2026/0355213 A1
(43) Pub. Date: Nov. 12, 2026

(54) TREATMENT OF INFECTIONS CAUSED BY INTRACELLULAR PATHOGENS

(71) Applicants: Sheba Impact Ltd., Ramat Gan (IL); Yeda Research and Development Co. Ltd., Rehovot (IL)

(72) Inventors: Ohad GAL-MOR, Hod-HaSharon (IL); Boaz ADANI, Herzliya (IL); David MARGULIS, Rehovot (IL); Leila MOTIEI, Rehovot (IL); Barr HAIM, Rehovot (IL); Shurrush KHRIESTO, Rehovot (IL)

(73) Assignees: Sheba Impact Ltd., Ramat Gan (IL); Yeda Research and Development Co. Ltd., Rehovot (IL)

(21) Appl. No.: 19/434,327

(22) Filed: Dec. 29, 2025

Related U.S. Application Data
(63) Continuation of application No. PCT/IL2024/050636, filed on Jun. 28, 2024.
(60) Provisional application No. 63/523,687, filed on Jun. 28, 2023.

(51) Int. Cl. A61K 31/55; A61K 31/138; A61K 31/351; A61K 31/42; A61K 31/4439; A61K 31/47; A61K 31/517; A61P 31/04

(52) U.S. Cl. CPC A61K 31/55; A61K 31/138; A61K 31/351; A61K 31/42; A61K 31/4439; A61K 31/47; A61K 31/517; A61P 31/04

(57) ABSTRACT

Compositions and methods for treating or preventing an infection by an intracellular pathogen in a subject in need thereof are provided.

### RELATED APPLICATIONS

This application is a Continuation of PCT Patent Application No. PCT/IL2024/050636 having International filing date of Jun. 28, 2024 which claims the benefit of priority under 35 USC § 119(e) of U.S. Provisional Patent Application No. 63/523,687 filed on Jun. 28, 2023. The contents of the above applications are all incorporated by reference as if fully set forth herein in their entirety.

### FIELD AND BACKGROUND OF THE INVENTION

The present invention, in some embodiments thereof, relates to treatment of infections caused by intracellular pathogens.

Infectious diseases still pose a major threat to both human and animal populations. A recent study estimated that infections by antibacterial resistant pathogens claim 700,000 lives every year globally, with 10 million projected deaths in the year 2050². Moreover, the WHO and World Economic Forum recently declared that antibacterial resistance is the greatest risk to human health. Even now, many infectious diseases are difficult to treat, resulting in high-dose administration of antibacterials, unbearable toxicity, delay in effective treatment, and increased mortality due to multidrug resistant (MDR) infections³. Importantly, this challenge is specifically prominent in the context of infections by intracellular bacterial pathogens. Intracellular bacterial pathogens invade host cells to establish an adapted replication niche, which facilitates their survival and dissemination. This compartment offers protection from the host's humoral immunity, sequestration from neutrophils, and access to nutrients that may be scarce extracellularly^{4, 5}. As a result, intracellular bacterial pathogens have evolved to sophisticatedly manipulate host cells to access their preferred niches within targeted cells. After invasion, bacteria are contained within a plasma membrane-derived vacuole, such as phagosomes or endosomes. Vacuolar intracellular bacteria, like Salmonella enterica or Mycobacterium tuberculosis remain within modified vacuoles, while intracellular cytosolic bacteria, like Listeria monocytogenes or Shigella spp., rupture the vacuole and reside in the host cytosol⁴⁻⁷.

Intracellular pathogens have co-evolved with hosts and developed a striking ability to manipulate and subvert multiple host functions and pathways to facilitate their survival and transmission. In general, the process by which intracellular bacteria hijack host cells can be divided into four distinct stages: adhesion, internalization, survival/proliferation, and dissemination. Although different pathogens use distinct strategies to subvert host pathways, eliminating the replicative niche of pathogens by regulated cell death of host cells serves as a universal defense mechanism against a wide array of intracellular pathogens. Pathogen-associated molecular patterns (PAMPs) are sensed by pattern-recognition receptors (PRRs), leading to the activation of regulated lytic cell death, including pyroptosis and necroptosis, and a proinflammatory response.^{5, 8, 9} This lytic form of cell death removes the replicative niche for intracellular pathogens by inducing the formation of large pores in the plasma membrane. This process releases highly inflammatory cytoplasmic contents, including proinflammatory cytokines and damage-associated molecular patterns (DAMPs), which recruit and activate immune cells to combat the infection¹⁰.

Although significant progress has been made in the last decades in our understanding of how intracellular pathogens interact with their hosts¹¹, this group of pathogens remains a major clinical concern worldwide, and the gained knowledge has not been translated into new therapeutic approaches yet. This challenge has become even more pressing with the increasing prevalence of MDR strains that are often linked to a more severe disease outcome and more resistant strains that continue to emerge worldwide¹⁰. Worrisomely, some of these bacteria are responsible for very important and common infectious diseases, including tuberculosis (TB), chlamydia, listeriosis, and invasive salmonellosis¹². Hence, there is an urgent need to develop novel and creative approaches to treat intracellular infections.

Additional background art includes:

Chandra et al. 2016 mSphere volume 1: issue 2 27.

### SUMMARY OF THE INVENTION

According to an aspect of some embodiments of the present invention there is provided a method of treating or preventing an infection by an intracellular pathogen in a subject in need thereof, the method comprising administering to the subject a therapeutically effective amount of a pharmaceutical composition comprising at least one compound represented by Formula I, II, III, IV, V, VI or VII, as described herein in any of the respective embodiments and any combination thereof.

According to an aspect of some embodiments of the present invention there is provided a method of inhibiting growth of an intracellular pathogen or invasion thereof into host cells, the method comprising contacting the pathogen with a therapeutically effective amount of a pharmaceutical composition comprising at least one compound represented by Formula I, II, III, IV, V, VI or VII as described herein in any of the respective embodiments and any combination thereof.

According to an aspect of some embodiments of the present invention there is provided a method of treating or preventing an infection by an intracellular pathogen in a subject in need thereof, the method comprising administering to the subject a therapeutically effective amount of a pharmaceutical composition comprising a compound which induces programmed cell death (PCD) in infected host cells of the intracellular pathogen, thereby treating or preventing the infection.

According to an aspect of some embodiments of the present invention there is provided a method of inhibiting growth of an intracellular pathogen or invasion thereof into host cells, the method comprising contacting the pathogen with a therapeutically effective amount of a pharmaceutical composition comprising a compound which induces PCD in host cells infected with an intracellular pathogen, thereby inhibiting growth of the intracellular pathogen or invasion thereof into host cells.

According to some embodiments of any of the embodiments described herein, the method is performed in vitro.

According to some embodiments of any of the embodiments described herein, the method is performed in vivo.

According to an aspect of some embodiments of the present invention there is provided a method of identifying an agent against intracellular pathogens, the method comprising:
(a) providing host cells infected with an intracellular pathogen genetically modified to express a reporter molecule under a cis-acting regulatory element of the intracellular pathogen;
(b) contacting the host cells with an antibiotic to eliminate extracellular pathogens;
(c) subjecting the host cells to a treatment with an agent;
(d) measuring a reporter activity of the reporter molecule in presence and in absence of the agent, wherein a decrease in the reporter activity in the presence of the agent in comparison to an absence thereof is indicative that the agent can be used against intracellular pathogens.

According to some embodiments of any of the embodiments described herein, the cis-acting regulatory element is a promoter.

According to some embodiments of any of the embodiments described herein, the promoter comprises the ssek3 promoter.

According to some embodiments of any of the embodiments described herein, the method further comprises determining viability of the host cells in the presence of the agent in comparison to an absence thereof, and wherein substantially the same viability is indicative that the agent is safe.

According to some embodiments of any of the embodiments described herein, the host cells are epithelial cells.

According to an aspect of some embodiments of the present invention there is provided a pharmaceutical composition comprising at least one compound represented by Formula I, II, III, IV, V, VI or VII as described herein in any of the respective embodiments and any combination thereof, for use in treating or preventing an infection by an intracellular pathogen in a subject in need thereof.

According to an aspect of some embodiments of the present invention there is provided a pharmaceutical composition comprising a compound which induces PCD in host cells of an intracellular pathogen for use in treating or preventing an infection by the intracellular pathogen in a subject in need thereof.

According to some embodiments of any of the embodiments described herein, the intracellular pathogen is Mycobacterium tuberculosis, the compound is represented by Formula I, II, III, IV, V or VI, or Formula by VII, provided that the compound is not:

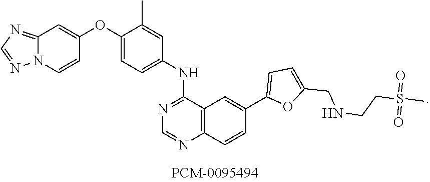 [SMILES: Cc1cc(Nc2ncnc3ccc(-c4ccc(CNCCS(C)(=O)=O)o4)cc23)ccc1Oc1ccn2ncnc2c1]

According to some embodiments of any of the embodiments described herein, the compound is not a Src inhibitor.

According to some embodiments of any of the embodiments described herein, the compound is not bactericidal.

According to some embodiments of any of the embodiments described herein, the intracellular pathogen is selected from the group consisting of bacterial pathogen, viral pathogen, fungal pathogen and a protozoan pathogen.

According to some embodiments of any of the embodiments described herein, the intracellular pathogen comprises bacteria.

According to some embodiments of any of the embodiments described herein, the bacteria is Gram positive.

According to some embodiments of any of the embodiments described herein, the bacteria is Gram negative.

According to some embodiments of any of the embodiments described herein, the bacteria is of a genus selected from the group consisting of Listeria monocytogenes, Salmonella enterica serovars (e.g., Salmonella Typhi), Mycobacterium tuberculosis, Chlamydia trachomatis, Rickettsia species and Legionella pneumophila.

According to some embodiments of any of the embodiments described herein, the bacteria comprises a vacuolar intracellular bacterium.

According to some embodiments of any of the embodiments described herein, the bacteria is Salmonella enterica or Mycobacterium tuberculosis.

According to some embodiments of any of the embodiments described herein, the bacteria comprises an intracellular cytosolic bacterium.

According to some embodiments of any of the embodiments described herein, the bacteria is Listeria monocytogenes or Shigella spp.

According to some embodiments of any of the embodiments described herein, the infection causes tuberculosis (TB), chlamydia, listeriosis, invasive salmonellosis, legionnaire's disease, Rocky Mountain spotted fever (R. rickettsii) or typhus (R. prowazekii).

According to some embodiments of any of the embodiments described herein, the compound is presented by Formula I:

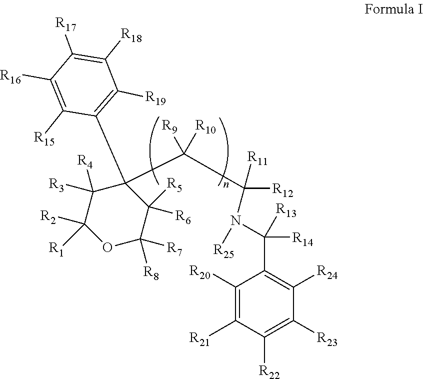

wherein:
n is 0 or 1;
R₁ and R₂ are each independently selected from hydrogen and alkyl, preferably a lower alkyl of 1 to 4 carbon atoms in length, wherein at least one of R₁ and R₂ is the alkyl;
R₃-R₁₄ are each independently selected from hydrogen, alkyl, cycloalkyl, halo, haloalkyl, hydroxy, alkoxy, thiol, thioalkoxy, amine, cyano and nitro;
R₁₅-R₂₄ are each independently selected from hydrogen, alkyl, cycloalkyl, amine, halo, hydroxy, thiol, alkoxy, and thioalkoxy; and
R₂₅ is selected from hydrogen, alkyl and cycloalkyl.

According to some embodiments of any of the embodiments described herein, n is 1.

According to some embodiments of any of the embodiments described herein, R₁ and R₂ are each independently selected from methyl, ethyl, propyl and isopropyl.

According to some embodiments of any of the embodiments described herein, R₁ and R₂ are each methyl; or R₁ is methyl and R₂ is ethyl; or R₁ is isopropyl and R₂ is hydrogen.

According to some embodiments of any of the embodiments described herein, R₃-R₁₃ are each hydrogen.

According to some embodiments of any of the embodiments described herein, R₁₄ is alkyl, preferably a lower alkyl (e.g., methyl).

According to some embodiments of any of the embodiments described herein, each of R₁₅-R₁₉ is hydrogen.

According to some embodiments of any of the embodiments described herein, at least one of R₁₅-R₁₉, preferably at least one of R₁₅, R₁₇ or R₁₉ is selected from alkyl (preferably lower alkyl), fluoro, and alkoxy (preferably lower alkoxy).

According to some embodiments of any of the embodiments described herein, R₁₅ is alkoxy (preferably lower alkoxy).

According to some embodiments of any of the embodiments described herein, at least one of R₂₀-R₂₄, preferably at least one of R₂₀, R₂₂ or R₂₄ is selected from amine (preferably a tertiary amine), fluoro, and alkoxy (preferably lower alkoxy).

According to some embodiments of any of the embodiments described herein, R₂₂ is selected from amine (preferably a tertiary amine), fluoro and alkoxy (preferably lower alkoxy).

According to some embodiments of any of the embodiments described herein, the compound is:

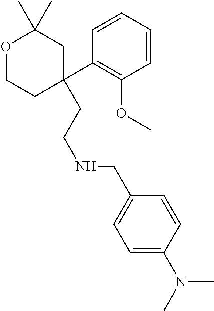 [SMILES: COc1ccccc1C1(CCNCc2ccc(N(C)C)cc2)CCOC(C)(C)C1]

PCM-1013431 (also referred to herein as C4).

According to some embodiments of any of the embodiments described herein, the compound is selected from compounds denoted as 21, 30, 46 and 50 (see, hereinbelow and FIG. 10B).

According to some embodiments of any of the embodiments described herein, the compound is selected from compounds denoted as 12, 15, 21, 24, 27, 28, 29, 30, 31, 46, 47, 48, 50, 51 and 53 (see, hereinbelow and FIG. 10B).

According to some embodiments of any of the embodiments described herein, the compound is represented by Formula II:

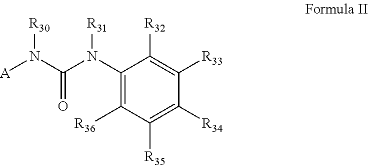

wherein:
R₃₀ and R₃₁ are each independently selected from hydrogen, alkyl and cycloalkyl;
R₃₂-R₃₆ are each independently selected from hydrogen, alkyl, cycloalkyl, halo, haloalkyl, hydroxy, alkoxy, thiol, thioalkoxy, amine, cyano and nitro, provided that at least one of R₃₂-R₃₆, preferably at least one of R₃₂, R₃₄ and R₃₆ is halo; and
A is selected from:

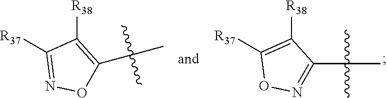

wherein the curved line represents an attachment point to NR₃₀₋; and R₃₇ and R₃₈ are each independently selected from hydrogen alkyl and cycloalkyl,

According to some embodiments of any of the embodiments described herein, R₃₀ and R₃₁ are each hydrogen.

According to some embodiments of any of the embodiments described herein, A is

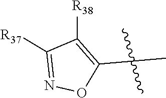

and at least two of R₃₂-R₃₆, preferably at least two of R₃₂, R₃₄ and R₃₆, are each halo, preferably chloro.

According to some embodiments of any of the embodiments described herein, A is

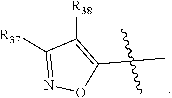

two of R₃₂, R₃₄ and R₃₆, are each halo, preferably chloro and the third is hydrogen, and R₃₃ and R₃₅ are each hydrogen.

According to some embodiments of any of the embodiments described herein, A is

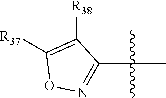

and no more than one of R₃₂-R₃₆, preferably one of R₃₂, R₃₄ and R₃₆, is halo, preferably chloro.

According to some embodiments of any of the embodiments described herein, A is

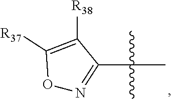

R₃₂ is halo, preferably chloro, and R₃₃-R₃₆ are each hydrogen.

According to some embodiments of any of the embodiments described herein, R₃₇ and R₃₈ are each independently selected from hydrogen and alkyl.

According to some embodiments of any of the embodiments described herein, the compound is selected from:

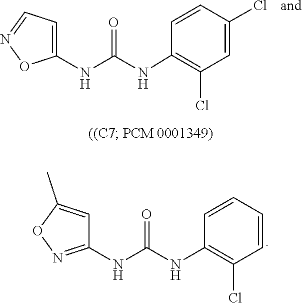 [SMILES: O=C(Nc1ccno1)Nc1ccc(Cl)cc1Cl ; "and" ; Cc1cc(NC(=O)Nc2ccccc2Cl)no1]

According to some embodiments of any of the embodiments described herein, the compound is represented by Formula III:

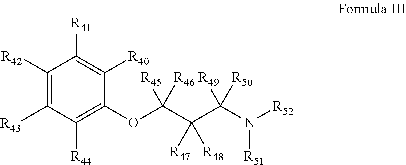

wherein:
R₄₀-R₄₄ are each independently selected from hydrogen, alkyl, cycloalkyl, halo, hydroxy, alkoxy, thiol, thioalkoxy, amine, provided that at least one of R₄₀-R₄₄, preferably R₄₀, is a cycloalkyl; and
R₄₅-R₅₂ are each independently selected from hydrogen, alkyl, cycloalkyl, halo, hydroxy, alkoxy, thiol, thioalkoxy, amine, provided that at least one of R₄₅-R₅₀ is selected from hydroxy, thiol, alkoxy and thioalkoxy,

According to some embodiments of any of the embodiments described herein, R₄₀ is a cycloalkyl and is preferably cyclopentyl.

According to some embodiments of any of the embodiments described herein, R₄₁-R₄₄ are each hydrogen.

According to some embodiments of any of the embodiments described herein, R₄₇ is hydroxy.

According to some embodiments of any of the embodiments described herein, R₄₅, R₄₆ and R₄₈-R₅₀ are each hydrogen.

According to some embodiments of any of the embodiments described herein, R₅₁ is hydrogen.

According to some embodiments of any of the embodiments described herein, R₅₂ is hydrogen or an alkyl, preferably a bulky lower alkyl such as t-butyl.

According to some embodiments of any of the embodiments described herein, the compound is:

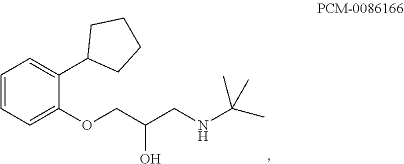 [SMILES: CC(C)(C)NCC(O)COc1ccccc1C1CCCC1]

and is also referred to herein as C2.

According to some embodiments of any of the embodiments described herein, the compound is represented by Formula IV:

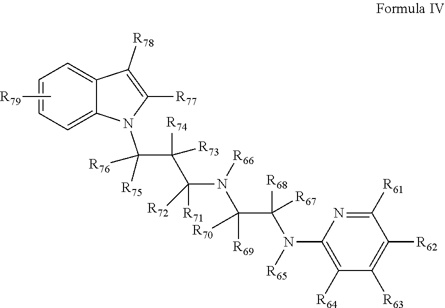

wherein:
R₆₁-R₆₄ are each independently selected from hydrogen, alkyl, cycloalkyl, halo, haloalkyl, amine, hydroxy, alkoxy, thiol, thioalkoxy;
R₆₅ and R₆₆ are each independently selected from hydrogen, alkyl and cycloalkyl;
R₆₇-R₇₈ are each independently selected from hydrogen, alkyl, cycloalkyl, halo, haloalkyl, amine, hydroxy, alkoxy, thiol and thioalkoxy; and
R₈₀ represents one or more (1, 2, 3, 4 or 5) substituents on the phenyl ring, or is absent, wherein each of these substituents, if present, is independently selected from alkyl, cycloalkyl, halo, haloalkyl, amine, hydroxy, alkoxy, thiol, and thioalkoxy,

According to some embodiments of any of the embodiments described herein, at least one of R₆₁-R₆₄, preferably R₆₄, is selected from alkyl and halo, and is preferably a trihaloalkyl.

According to some embodiments of any of the embodiments described herein, each of R₆₅ and R₆₆ is hydrogen.

According to some embodiments of any of the embodiments described herein, R₆₇-R₇₀ are each hydrogen.

According to some embodiments of any of the embodiments described herein, at least one of R₇₁-R₇₆, preferably R₇₄, is hydroxy.

According to some embodiments of any of the embodiments described herein, R₇₁-R₇₃, R₇₅ and R₇₆ are each hydrogen and R₇₄ is hydroxy.

According to some embodiments of any of the embodiments described herein, R₇₇ and R₇₈ are each hydrogen and/or R₇₉ is absent.

According to some embodiments of any of the embodiments described herein, the compound is:

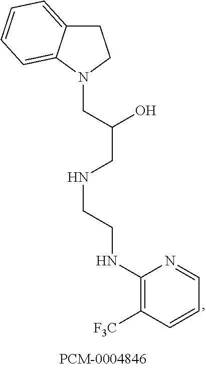 [SMILES: OC(CNCCNc1ncccc1C(F)(F)F)CN1CCc2ccccc21]

which is also referred to herein as C3.

According to some embodiments of any of the embodiments described herein, the compound is represented by Formula V:

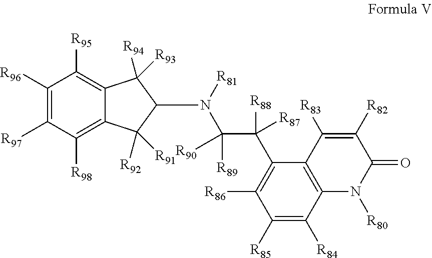

wherein:
R₈₀ and R₈₁ are each independently selected from hydrogen, alkyl and cycloalkyl;
R₈₂-R₈₆ are each independently selected from hydrogen, alkyl, cycloalkyl, halo, haloalkyl, amine, hydroxy, alkoxy, thiol, and thioalkoxy;
R₈₇-R₉₀ are each independently selected from hydrogen, alkyl, cycloalkyl, halo, haloalkyl, amine, hydroxy, alkoxy, thiol, and thioalkoxy;
R₉₁-R₉₄ are each independently selected from hydrogen, alkyl, cycloalkyl, halo, haloalkyl, amine, hydroxy, alkoxy, thiol, and thioalkoxy; and
R₉₅-R₉₈ are each independently selected from hydrogen, alkyl, cycloalkyl, halo, haloalkyl, amine, hydroxy, alkoxy, thiol, and thioalkoxy,

According to some embodiments of any of the embodiments described herein, R₈₀ and R₈₁ are each hydrogen.

According to some embodiments of any of the embodiments described herein, R₈₂ and R₈₃ are each hydrogen.

According to some embodiments of any of the embodiments described herein, at least one of R₈₄-R₈₆, preferably R₈₄, is hydroxy.

According to some embodiments of any of the embodiments described herein, at least one of R₈₇-R₉₀, preferably R₈₇, is hydroxy.

According to some embodiments of any of the embodiments described herein, each of R₉₁-R₉₄ is hydrogen.

According to some embodiments of any of the embodiments described herein, at least one, or at least two of R₉₅-R₉₈ is an alkyl, preferably a lower alkyl.

According to some embodiments of any of the embodiments described herein, R₉₆ and R₉₇ are each independently an alkyl, preferably a lower alkyl.

According to some embodiments of any of the embodiments described herein, the compound is:

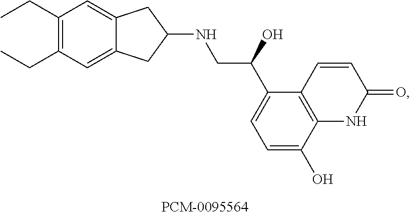 [SMILES: CCc1cc2c(cc1CC)CC(NC[C@@H](O)c1ccc(O)c3[nH]c(=O)ccc13)C2]

also referred to herein as C8.

According to some embodiments of any of the embodiments described herein, the compound is represented by Formula VI:

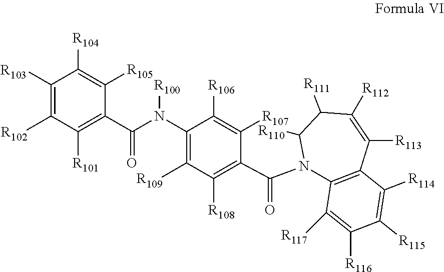

wherein:
R₁₀₀ is hydrogen, alkyl or cycloalkyl;
R₁₀₁-R₁₀₅ are each independently selected from hydrogen, alkyl, cycloalkyl, aryl, halo, haloalkyl, amine, hydroxy, alkoxy, thiol, thioalkoxy, heteroaryl, cyano, and nitro, wherein at least one of R₁₀₁-R₁₀₅, preferably R₁₀₁, is aryl;
R₁₀₆-R₁₀₉ are each independently selected from hydrogen, alkyl, cycloalkyl, halo, haloalkyl, amine, hydroxy, alkoxy, thiol and thioalkoxy;
R₁₁₀-R₁₁₃ are each independently selected from hydrogen, alkyl, cycloalkyl, halo, haloalkyl, amine, hydroxy, alkoxy, thiol and thioalkoxy, or, alternatively, two of R₁₁₀-R₁₁₃ form together a substituted or unsubstituted cyclic ring (alicyclic, heteroalicyclic, aryl or heteroaryl); and
R₁₁₄-R₁₁₇ are each independently selected from hydrogen, alkyl, cycloalkyl, halo, haloalkyl, amine, hydroxy, alkoxy, thiol and thioalkoxy,

According to some embodiments of any of the embodiments described herein, R₁₀₀ is hydrogen.

According to some embodiments of any of the embodiments described herein, at least one of R₁₀₁-R₁₀₅ is aryl, preferably phenyl.

According to some embodiments of any of the embodiments described herein, R₁₀₁ is the aryl, and wherein optionally R₁₀₂-R₁₀₅ are each hydrogen.

According to some embodiments of any of the embodiments described herein, at least two of R₁₁₀-R₁₁₃ form together a substituted or unsubstituted cyclic ring, preferably a substituted or unsubstituted heteroaryl.

According to some embodiments of any of the embodiments described herein, R₁₁₂ and R₁₁₃ form together a substituted or unsubstituted imidazole.

According to some embodiments of any of the embodiments described herein, R₁₀₆-R₁₀₉ are each hydrogen and/or R₁₁₄-R₁₁₇ are each hydrogen.

According to some embodiments of any of the embodiments described herein, the compound is:

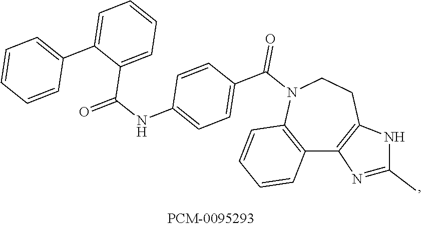 [SMILES: Cc1nc2c([nH]1)CCN(C(=O)c1ccc(NC(=O)c3ccccc3-c3ccccc3)cc1)c1ccccc1-2]

also referred to herein as C6.

According to some embodiments of any of the embodiments described herein, the compound is represented by Formula VII:

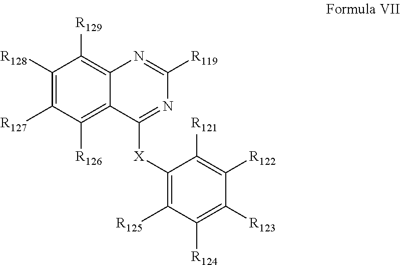

wherein:
X is O, S or NR₁₂₀;
R₁₁₉ and R₁₂₀ (if present) are each independently selected from hydrogen, alkyl or cycloalkyl;
R₁₂₁-R₁₂₅ are each independently selected from hydrogen, alkyl, cycloalkyl, aryl, halo, haloalkyl, amine, hydroxy, alkoxy, aryloxy, thioaryloxy, thiol, thioalkoxy, heteroaryl, cyano and nitro, or, alternatively or in addition, two of R₁₂₁-R₁₂₅ form together a substituted or unsubstituted cyclic ring (alicyclic, heteroalicyclic, aryl or heteroaryl); and
R₁₂₆-R₁₂₉ are each independently selected from hydrogen, alkyl, cycloalkyl, halo, haloalkyl, amine, hydroxy, alkoxy, thiol, thioalkoxy, heteroalicyclic, heteroaryl, and aryloxy, provided that at least one of R₁₂₁-R₁₂₅ and at least one of R₁₂₆-R₁₂₉ is other than hydrogen.

According to some embodiments of any of the embodiments described herein, X is NR₁₂₀.

According to some embodiments of any of the embodiments described herein, R₁₁₉ and R₁₂₀, if present, are each hydrogen.

According to some embodiments of any of the embodiments described herein, at least one of R₁₂₁-R₁₂₅ is alkoxy or aryloxy.

According to some embodiments of any of the embodiments described herein, at least one of R₁₂₆-R₁₂₉ is alkoxy, aryloxy or a heteroaryl.

According to some embodiments of any of the embodiments described herein, at least two of R₁₂₁-R₁₂₅ form together a cyclic ring, preferably a heteroalicyclic ring.

According to some embodiments of any of the embodiments described herein, R₁₂₁ and R₁₂₂ form together the cyclic ring.

According to some embodiments of any of the embodiments described herein, at least two of R₁₂₆-R₁₂₉ are each independently an alkoxy.

According to some embodiments of any of the embodiments described herein, at least two of R₁₂₆-R₁₂₉ are each independently an alkoxy.

According to some embodiments of any of the embodiments described herein, the compound is represented by Formula VIIa:

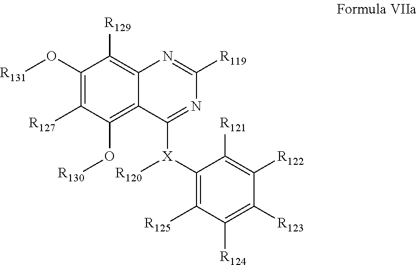

wherein:
R₁₂₁ and R₁₂₂ form together the cyclic ring, preferably the heteroalicyclic ring; and
R₁₃₀ and R₁₃₁ are each independently selected from alkyl, cycloalkyl, and heteroalicyclic.

According to some embodiments of any of the embodiments described herein, R₁₂₁ and R₁₂₂ form together a dioxolane.

According to some embodiments of any of the embodiments described herein, at least one of R₁₂₃-R₁₂₅, preferably R₁₂₅, is halo, preferably chloro.

According to some embodiments of any of the embodiments described herein, R₁₃₀ and R₁₃₁ each independently comprise a heteroalicyclic.

According to some embodiments of any of the embodiments described herein, R₁₃₀ is a heteroalicyclic, preferably tetrahydropyrane.

According to some embodiments of any of the embodiments described herein, R₁₃₁ is an alkyl substituted by a heteroalicylic, preferably a piperazine.

According to some embodiments of any of the embodiments described herein, the compound is:

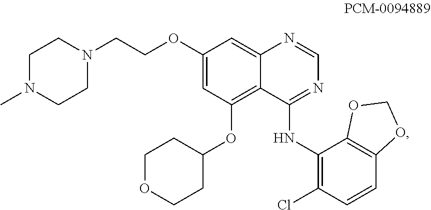 [SMILES: CN1CCN(CCOc2cc(OC3CCOCC3)c3c(Nc4c(Cl)ccc5c4OCO5)ncnc3c2)CC1]

and is also referred to herein as C1.

According to some embodiments of any of the embodiments described herein, at least one of R₁₂₁-R₁₂₅ is aryloxy, preferably heteroaryloxy.

According to some embodiments of any of the embodiments described herein, R₁₂₃ is the aryloxy.

According to some embodiments of any of the embodiments described herein, at least one of R₁₂₆-R₁₂₉ is a heteroaryl, preferably furyl, more preferably a substituted furyl.

According to some embodiments of any of the embodiments described herein, R₁₂₇ is the heteroaryl.

According to some embodiments of any of the embodiments described herein, the compound is represented by Formula VIIb:

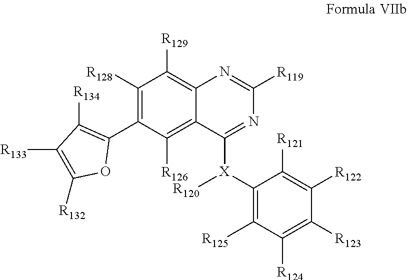

wherein:
at least one of R₁₂₁-R₁₂₅ is the aryloxy, preferably a heteroaryloxy; and
R₁₃₂-R₁₃₄ are each independently selected from hydrogen, alkyl, cycloalkyl, halo, haloalkyl, amine, heteroalicyclic, heteroaryl, cyano and nitro.

According to some embodiments of any of the embodiments described herein, R₁₂₃ is heteroaryloxy, preferably 7-triazolopyridinoxy.

According to some embodiments of any of the embodiments described herein, at least one of R₁₂₁, R₁₂₂, R₁₂₄ and R₁₂₅, preferably R₁₂₄, is alkyl, preferably methyl.

According to some embodiments of any of the embodiments described herein, at least one of R₁₃₂-R₁₃₄, preferably R₁₃₂, is an alkyl, preferably a substituted alkyl.

According to some embodiments of any of the embodiments described herein, the alkyl is substituted by a moiety that comprises an amine and/or SO₂, preferably a hydrocarbon interrupted and/or substituted by an amine and/or SO₂.

According to some embodiments of any of the embodiments described herein, the alkyl is —CH₂—NH—CH₂—CH₂—SO₂—CH₃.

According to some embodiments of any of the embodiments described herein, the compound is:

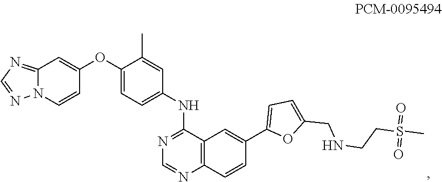 [SMILES: Cc1cc(Nc2ncnc3ccc(-c4ccc(CNCCS(C)(=O)=O)o4)cc23)ccc1Oc1ccn2ncnc2c1]

also referred to herein as C5.

According to some embodiments of any of the embodiments described herein, R₁₂₃ is other than heteroaryloxy; and/or R₁₃₂ is other than an alkyl substituted by a moiety that comprises an amine and/or SO₂, or a hydrocarbon interrupted and/or substituted by an amine and/or SO₂.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an illustration showing an embodiment of high-throughput screening (HTS) for compounds having activity against intracellular pathogens.

FIGS. 2A-B present a scheme showing an embodiment of the pipeline and selection stages of the HTS illustrated in FIG. 1 (FIG. 2A) and the chemical structures of exemplary top hits identified in the HTS (FIG. 2B);

FIG. 3 shows an inhibitory effect of compounds identified in the HTS on Salmonella infection;

FIG. 4 shows the cytotoxic activity (against HeLa and HB2 cells) of compounds identified in the HTS;

FIG. 5 shows the inhibitory activity of compounds identified in the HTS in a dose-response assay;

FIG. 6 shows that compounds identified in the HTS inhibit mainly Salmonella intracellular replication rather than invasion;

FIG. 7 shows activity of compounds identified in the HTS in inhibiting intracellular growth of S. Typhimurium in a mouse bone-marrow derived macrophages (BMDMs);

FIG. 8 shows that compounds identified in the HTS inhibit the intracellular growth of Listeria monocytogenes; and

FIG. 9 shows that compounds identified in the HTS trigger programmed cell death in infected host cells;

FIGS. 10A-B present data obtained in Structure Activity Relationship (SAR) studies of compounds PCM-0103431 and PCM-0001349 (FIG. 10A) and the chemical structures of exemplary structural analogs used in the SAR studies (FIG. 10B).

### DESCRIPTION OF SPECIFIC EMBODIMENTS OF THE INVENTION

The present invention, in some embodiments thereof, relates to treatment of infections caused by intracellular pathogens.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Intracellular bacterial pathogens including Mycobacterium, Listeria, and Salmonella continue to threaten human and animal health globally, affecting millions of lives and causing substantial economic loss. The ability of these clinically important pathogens to invade and localize within host cells provide them protection from both antibacterials and the host immune system, which can lead to chronic untreatable infections. Furthermore, the constant emergence and spreading of new multi- and extreme-drug resistant strains, significantly limit and often completely prevent antibiotic therapy. Thus, novel approaches and new treatment options are urgently required to fight intracellular resistant pathogens.

Whilst conceiving embodiments of the invention and reducing them to practice, the present inventors devised a novel high throughput screening approach for the identification of non-toxic, not bactericidal compounds that can be used for combatting intracellular pathogens.

As is described hereinbelow and in the Examples section which follows, the present inventors applied a multistage high-throughput screening (HTS) of nearly 40,000 compounds and successfully identified a number of non-bactericidal, non-toxic, active molecules that effectively inhibit the intracellular growth of pathogens like Salmonella in epithelial and phagocytic human host cells at μM concentrations. Importantly, such compounds inhibit intracellular growth of Listeria as well. Structure activity relationship (SAR) studies that were conducted for selected compounds identified chemical derivatives with an improved activity against intracellular infection of pathogens. It is suggested that the present methodology and compounds identified thereby act as host-directed therapies (HDTs) and as such pave the way for the identification of the next generation therapy against intracellular bacterial pathogens.

In contrast to conventional antibiotics that directly act on bacteria, it is suggested that the identified compounds act as host-directed therapies HDTs that enhance a potentially broad response against bacteria, and render host cells less-permissive for bacterial growth. Notably, HDTs have several important advantages over conventional antibiotic therapy, including: (i) HDTs can be effective against MDR infections; (ii) unlike antibiotics that directly target bacteria, HDTs act against intracellular pathogens by either potentiating host defenses or sensitizing internalized bacteria to immune clearance; (iii) in absence of direct selective pressure on the pathogen, HDT are less likely to develop drug resistance; (iv) HDTs have the potential to target metabolically inactive, non-replicating bacteria, which are tolerant to conventional therapies; and (v) potential synergies with HDTs and antibiotics acting on different pathways. Indeed, recent advances in HDTs have provided promising approaches to reduce or eliminate intracellular bacterial infections, by targeting the host factors, that can restrict the replication and persistence of pathogens inside the cell.

The fact that the discovered compounds are not bactericidal provides a significant advantage compared to conventional antibiotics in the sense that there is much lower chance for the development of resistance against these compounds by the pathogens, and that these compounds are not expected to disrupt the natural microbiota, which are important component for the human health.

Thus, according to an aspect of the invention, there is provided a method of treating or preventing an infection by an intracellular pathogen in a subject in need thereof, the method comprising administering to the subject a therapeutically effective amount of a pharmaceutical composition comprising at least one compound represented by Formula I, II, III, IV, V, VI or VII, as described herein in any of the respective embodiments and any combination thereof.

Alternatively, or additionally, there is provided a method of inhibiting growth of an intracellular pathogen or invasion thereof into host cells, the method comprising contacting the pathogen with a therapeutically effective amount of a pharmaceutical composition comprising at least one compound represented by Formula I, II, III, IV, V, VI or VII, as described herein in any of the respective embodiments and any combination thereof.

As used herein the phrase “inhibiting the growth of an intracellular pathogen” refers to inhibiting survival and/or replication of an intracellular pathogen during host cell infection.

As used herein the phrase “inhibiting invasion to host cells” refers to inhibiting the entry or penetration of the pathogen into the host cell. This can be determined by a bioluminescence assay, microscopy, CFU counting and by other methods. The inventors have developed a bioluminescence-based method, in which the pathogen expresses a reporter luciferase system under the control of a regulatory element that is induced only when the pathogen is in the host cell (but not in the extracellular environment), the intensity of the bioluminescence signal is proportionate to the amount of the intracellular pathogen. That is, the more pathogen invades, survives or replicate intracellularly, the higher the intensity of the bioluminescence signal.

As used herein “inhibiting” refers to a decrease of at least 10%, 20%, 30%, 40%, 50%, 60%, 70%, 80%, 90%, 95% or complete inhibition (i.e., 100%) in the survival, growth, or replication of the intracellular pathogen or invasion thereof as compared to that of an identical pathogen in an identical host system under the same conditions but without the compound or agent.

Alternatively, or additionally, there is provided a method of treating or preventing an infection by an intracellular pathogen in a subject in need thereof, the method comprising administering to the subject a therapeutically effective amount of a pharmaceutical composition comprising a compound which induces programed cell death in host cells infected by an intracellular pathogen, thereby treating or preventing the infection.

As used herein “programed cell death” abbreviated as PCD refers to caspase dependent cell death (such as apoptosis and pyroptosis) and non-caspase dependent cell death (such as necroptosis, ferrotosis, and autophagy). Without being bound by theory, it is suggested that the selective activation of caspase 3 and caspase 8, but not caspase 9 leads to a caspase-dependent cell death that can be used to control infection.

As used herein “induction” refers to an increase of at least 10%, 20%, 30%, 40%, 50%, 60%, 70%, 80%, 90%, 95% or more (e.g., at least 2, 3, 5, 10 fold) in a PCD signal in response to the compound or agent as compared to that in the absence thereof.

Alternatively or additionally, there is provided a method of inhibiting growth of an intracellular pathogen or invasion thereof into host cells, the method comprising contacting the pathogen with a therapeutically effective amount of a pharmaceutical composition comprising a compound which induces PCD in host cells of the intracellular pathogen, thereby inhibiting growth of the intracellular pathogen or invasion thereof into host cells.

Alternatively or additionally, there is provided a pharmaceutical composition comprising at least one compound represented by Formula I, II, III, IV, V, VI or VII, as described herein in any of the respective embodiments and any combination thereof for use in treating or preventing an infection by an intracellular pathogen in a subject in need thereof.

Alternatively or additionally, there is provided a pharmaceutical composition comprising a compound which induces PCD in host cells of an intracellular pathogen for use in treating or preventing an infection by the intracellular pathogen in a subject in need thereof.

As used herein, the term “treating” refers to curing, reversing, attenuating, alleviating, minimizing, suppressing or halting the deleterious effects of a pathogen infection.

As used herein, the term “preventing” refers to preventing pathogen infection or the deleterious effects (e.g., symptoms) of a pathogen infection.

The terms “administer,” “administering,” or “administration” refers to injecting, implanting, absorbing, ingesting, or inhaling a compound or agent described herein, or a pharmaceutical composition thereof.

Subjects which may be treated according to this aspect of the present invention include animal subjects—e.g. humans; or any mammalian host; or plants which may suffer from a disease associate with infection by intracellular pathogens.

According to one embodiment, the infection is an acute infection.

According to another embodiment, the infection is a chronic infection.

As used herein “intracellular pathogen” refers to a pathogen that may belong to a diverse range of life forms including bacteria, viruses, fungi, protozoa, and parasitic worms that cause disease to a host infected therewith. Facultative or obligatory intracellular pathogens are characterized by their ability to invade and reside within host cells, often evading the host immune system and causing various diseases.

Following are some non-limiting examples of intracellular pathogens:
Intracellular Bacteria (can be Gram positive or Gram negative): Listeria monocytogenes: Causes listeriosis. Invades and replicates within macrophages and other host cells, spreading cell-to-cell; Salmonella enterica (e.g., Salmonella enterica serovar Typhi): Causes typhoid fever and salmonellosis. Survives and multiplies within macrophages and epithelial cells; Mycobacterium tuberculosis: Causes tuberculosis. Persists within macrophages by inhibiting phagosome-lysosome fusion; Chlamydia trachomatis: Causes chlamydia. Obligate intracellular pathogen that replicates within a specialized vacuole inside host cells; Rickettsia species: Causes diseases like Rocky Mountain spotted fever (R. rickettsii) or typhus (R. prowazekii).
Legionella pneumophila: Causes Legionnaires' disease. Survives and replicates within amoebae in the environment and macrophages in humans.

According to a specific embodiment, the bacteria is of a genus selected from the group consisting of Listeria monocytogenes, Salmonella enterica (e.g., Salmonella enterica serovar Typhi), Mycobacterium tuberculosis, Chlamydia trachomatis, Rickettsia species and Legionella pneumophila.

According to a specific embodiment, the bacteria reside within an intracellular vacuoles containing bacteria.

According to a specific embodiment, the bacteria is Salmonella enterica or Mycobacterium tuberculosis.

According to a specific embodiment, the bacteria reside intracellularly in its host cells cytosol.

According to a specific embodiment, the bacteria is Listeria monocytogenes or Shigella spp.

According to a specific embodiment, the infection causes tuberculosis (TB), chlamydia, listeriosis, invasive salmonellosis, legionnaire's disease, Rocky Mountain spotted fever (R. rickettsii) or typhus (R. prowazekii).

Intracellular Viruses: It will be appreciated that all viruses are intracellular pathogens as they require host cell machinery for replication. Following are some examples: Human Immunodeficiency Virus (HIV): Causes AIDS. Infects CD4+ T cells, macrophages, and dendritic cells; Influenza virus: Causes the flu. Infects respiratory epithelial cells; Hepatitis B virus (HBV): Causes hepatitis B. Infects liver cells (hepatocytes); Herpes Simplex Virus (HSV): Causes oral and genital herpes.

Infects epithelial cells and establishes latency in neurons; Varicella-Zoster Virus (VZV): Causes chickenpox and shingles; Infects skin and nerve cells. Coronavirus: e.g., SARS-CoV2 causes various forms of COVID19. Infects respiratory epithelial cells, endothelial cells, gastrointestinal cells and more.

Intracellular Fungi: Histoplasma capsulatum: Causes histoplasmosis. Survives within macrophages. Cryptococcus neoformans: Causes cryptococcosis, primarily in immunocompromised individuals. Can survive intracellularly in macrophages;

Intracellular Protozoa: Plasmodium species (e.g., Plasmodium falciparum): Causes malaria. Infects red blood cells and liver cells; Toxoplasma gondii: Causes toxoplasmosis. Invades and replicates within nucleated cells, especially in muscle and brain tissues; Leishmania species: Causes leishmaniasis. Infects macrophages; Trypanosoma cruzi: Causes Chagas disease. Infects various cell types, including muscle and nerve cells;

Parasitic Worms: Intracellular Helminths. While most helminths are extracellular, some have intracellular stages during their lifecycle; Trichinella spiralis:

Causes trichinosis. Encysts within muscle cells.

According to a specific embodiment, the virus is selected from the group consisting of Adeno-associated virus (AAV), Polyomavirus, SV40, Papillomavirus, Adeonovirus, Herpes simplex virus, EBV, VZV, Poliovirus, Rhinovirus, Hepatitis A, B, and C virus, Yellow fever virus, Influenza virus, Measles virus, Reovirus, Rotavirus virus, HIV, and SARS virus.

According to a specific embodiment, the protozoa is selected from the group consisting of Trypanosoma cruzi, Leishmania, Plasmodium, Polypodium, Cryptosporidium parvum, and Toxoplasma gondii.

According to a specific embodiment, the fungus is selected from the group consisting of Histoplasma capsulatum, Cryptococcus neoformans, Blastomyces dermatitidis, and Pneumocystis jirovecii.

According to a specific embodiment, the virus is selected from the group consisting of Salmonella, Chlamydia, Rickettsia, Coxiella, Mycobacterium, and listeria.

According to a specific embodiment, the intracellular pathogen is Mycobacterium tuberculosis, and the compound is represented by Formula I, II, III, IV, V or VI, or by VII, provided that the compound is not:

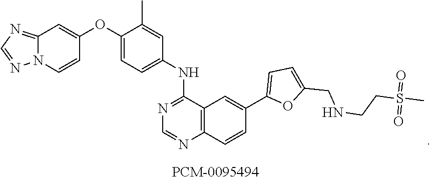 [SMILES: Cc1cc(Nc2ncnc3ccc(-c4ccc(CNCCS(C)(=O)=O)o4)cc23)ccc1Oc1ccn2ncnc2c1]

According to a specific embodiment, the intracellular pathogen is Mycobacterium tuberculosis, and the compound is represented by Formula I, II, III, IV, V or VI, or by Formula VIIa.

According to a specific embodiment, the compound is represented by Formula I, II, III, IV, V or VI, or by Formula VIIa.

According to a specific embodiment, the compound is not a Src inhibitor.

As used herein a “Src inhibitor” refers to a class of compounds (some are FDA approved drugs, e.g., Dasatinib, Bosutinib, Saracatinib (AZD0530) and KX2-391) that target and inhibit the activity of Src family kinases (SFKs). Src kinases are a group of non-receptor tyrosine kinases that play crucial roles in various cellular processes, including proliferation, differentiation, survival, and migration. Dysregulation of Src kinases has been implicated in the development and progression of several cancers and other diseases. Src inhibitors function by binding to the ATP-binding site of Src kinases, preventing their activation and subsequent phosphorylation of downstream targets.

According to a specific embodiment, the compound is not bactericidal.

As used herein “bactericidal” refers to refers to compounds or agents that kill bacteria, effectively eliminating bacterial populations rather than merely inhibiting their growth.

It will be appreciated that some of the compounds described herein have been identified as having activity against intracellular pathogens using an innovative screening tool that can be harnessed towards the identification of additional agent or compounds.

Thus, according to an aspect of the invention there is provided a method of identifying an agent against intracellular pathogens, the method comprising:
(a) providing host cells infected with an intracellular pathogen genetically modified to express a reporter system under a cis-acting regulatory element of the intracellular pathogen;
(b) contacting the host cells with an antibiotic to eliminate extracellular pathogens;
(c) subjecting the host cells to a treatment with an agent;
(d) measuring a reporter activity of the reporter molecule in presence and in absence of the agent, wherein a decrease in the reporter activity in the presence of the agent in comparison to an absence thereof is indicative that the agent can be used against intracellular pathogens.

As used herein “agent” refers to a chemical compound which can be naturally occurring or synthetic.

It is envisaged that the agent thus identified can be broad spectrum to inhibit the survival of any intracellular pathogens (or subgroup thereof, e.g., bacteria, e.g., vacuolar intracellular), such as when it acts on host or pathogen function that is critical for the ability of the pathogen to infect, survive or replicate within its host.

According to other embodiments, the agent thus identified is specific to the pathogen (at the level or the genus, species or strain) on which it was selected.

The method relies on the expression of a reporter molecule which is induced to be expressed from the pathogen only when it has invaded the host cell. At the extracellular environment its expression is generally null.

Thus, the pathogen is transfected or transformed with a plasmid that comprises a nucleic acid sequence encoding a reporter molecule under the regulation of a cis acting regulatory element active only in the intracellular environment.

The cis-acting regulatory element can be for example a promoter. This, for example a promoter of a gene that is induced intracellularly. Examples of such genes include but are not limited to the Salmonella pathogenicity island 2 genes (ssaR, ssrB, SifA, sseA etc.) and the PhoPQ regulon (phop, pagC, mgtA etc). In Listeria monocytogenes prfA, actA, hly, plcA, plcB and others. In Mycobacterium tuberculosis: phoP, sigE, esat-6, and cfp-10. For intracellular fungus Cryptococcus neoformans, examples include CAP10, FKS1, SOD1 and SOD2. For intracellular Histoplasma capsulatum the cbp1 and Yps3 are induced intracellularly. For the intracellular parasite Plasmodium, the var and PfEMP1 genes are induced during host cell infection. For viral pathogens such as the adeno-associated virus, the rep (replication) and cap (capsid) genes are induced during cell infection and can be used for the construction of the reporter system.

According to a specific embodiment, the promoter is the ssek3 promoter (see e.g., Brown et al. 2011 PLOS ONE 6(3):e17824).

The reporter gene can be for example any that emits a measurable or detectable signal such as a bioluminescent or fluorescent signal: fluorescent proteins (e.g., green/red/yellow fluorescent protein), lacZ (β-galactosidase), luciferase (lux, e.g., luxCDABE) as described in the Examples section which follows, CAT (Chloramphenicol Acetyltransferase) and GUS (β-glucuronidase).

Enhancer elements can stimulate transcription up to 1,000 fold from linked homologous or heterologous promoters. Enhancers are active when placed downstream or upstream from the transcription initiation site. These can be derived from the above genes as well.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Non-limiting examples of bacterial constructs include the pET series of E. coli expression vectors [Studier et al. (1990) Methods in Enzymol. 185:60-89) or pCS26 [

Usage of a Bioluminescence Reporter System to Image Promoter Activity During Host Infection. Aviv G, Gal-Mor O. Methods Mol Biol. 2018; 1734:33-38. PMID: 29288444].

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

The pathogen may be transformed stably or transiently with the nucleic acid constructs disclosed herein. In stable transformation, the nucleic acid molecule is integrated into the cell genome and as such it represents a stable and inherited trait. In transient transformation, the nucleic acid molecule is expressed by the cell transformed but it is not integrated into the genome and as such it represents a transient trait.

Once the transformed pathogen (i.e., genetically modified pathogen) is at hand it is used to transfect or infect the host.

The host organism can be plant or animal host (e.g., human, see Examples section).

Once the transformed cells are obtained, and the presence of the genetically modified pathogen therein is affirmed (by detecting the signal from the reporter molecule), it is subjected to treatment with the test agent.

Optionally prior to treatment with the agent or alternatively or additionally following treatment, antibiotics which acts extracellularly is added to the culture to eliminate extracellular pathogens. This allows measuring the effect of the examined agent on the invasion/survival/replication of the intracellular pathogen.

Following sufficient time to convey the effect of the test agent e.g., 1-24 hours (which can start before the addition of the pathogen e.g., 1-2 hours, concomitantly with or after its addition), the reporter activity is measured in comparison to a control sample which does not include the test agent. A decrease in the reporter activity in the presence of the agent in comparison to an absence thereof is indicative that the agent can be used against intracellular pathogens.

The assay can be done in large scale, testing many agents individually or in a pooled manner, in which different compounds are tested together.

Once effective agents are identified in the reporter assay, they can be further qualified, as anti-pathogenic agents, which are selective to the pathogen and not to the host cells.

Thus, according to a specific embodiment, the method further comprises determining viability of the host cells in the presence of the agent in comparison to an absence thereof, and wherein substantially the same viability (e.g., about 5-10%, 5-20%, 5-15%) is indicative that the agent is not-toxic to host cells.

According to a specific embodiment, any of the methods described herein or uses of the compounds or agents, may be performed in vitro, e.g., in cell models (e,g., as described in the Examples section which follows) or on surfaces, such as described hereinbelow.

According to a specific embodiment, any of the methods described herein or uses of the compounds or agents, may be performed in vivo (necessitating their administration to a human or animal host).

Thus, the compounds or agents described herein and/or derivative thereof (and optional additional anti-microbial agent) may be administered per se, or as part of a pharmaceutical composition.

The phrase “pharmaceutical composition”, as used herein refers to a preparation of one or more of the active ingredients described herein with other chemical components such as physiologically suitable carriers and excipients. The purpose of a pharmaceutical composition is to facilitate administration of a compound to an organism.

As used herein the term “active ingredient” refers to the agents of the present invention accountable for the intended biological effect.

Hereinafter, the phrases “physiologically acceptable carrier” and “pharmaceutically acceptable carrier”, which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences”, Mack Publishing Co., Easton, PA, which is herein fully incorporated by reference and are further described herein below.

It will be appreciated that the agents of the present invention can be provided to the individual with additional active agents to achieve an improved therapeutic effect as compared to treatment with each agent by itself.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

The preparation of the present invention may also be formulated as topical compositions, such as a spray, a cream, a mouthwash, a wipe, a foam, a soap, an oil, a solution, a lotion, an ointment, a paste and a gel.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compounds according to any of the embodiments described herein are collectively represented by Formula I, II, III, IV, V, VI or VII, as follows.

According to some embodiments of any of the embodiments described herein, the compound is represented by Formula I:

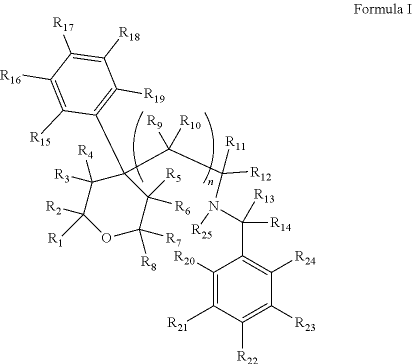

wherein:
n is 0, 1, 2, 3, 4, or a higher integer, preferably, 0, 1, or 2, and more preferably is 0 or 1;
R₁ and R₂ are each independently selected from hydrogen and alkyl;
R₃-R₁₄ are each independently selected from hydrogen, alkyl, cycloalkyl, halo, haloalkyl, hydroxy, alkoxy, thiol, thioalkoxy, amine, cyano and nitro, and from any of the other substituents as described herein;
R₁₅-R₂₄ are each independently selected from hydrogen, alkyl, cycloalkyl, amine, halo, hydroxy, thiol, alkoxy, and thioalkoxy; and
R₂₅ is selected from hydrogen, alkyl and cycloalkyl.

According to some embodiments of any of the embodiments described herein, n is 1. When n is 0, R₉ and R₁₀ are absent. When n is 2 or more, R₉ and R₁₀ in each CR₉R₁₀ moiety can be the same or different. In exemplary embodiments, R₉ and R₁₀, when present, are each hydrogen.

According to some embodiments of any of the embodiments described herein, at least one, and preferably each, of R₁ and R₂ is alkyl, and preferably a lower alkyl of, e.g., 1 to 6, or 1 to 4 carbon atoms in length. The alkyl can be linear or branched and can be substituted or substituted. In some embodiments, the alkyl is unsubstituted. In some embodiments, the alkyl is a linear unsubstituted lower alkyl as described herein.

According to some embodiments of any of the embodiments described herein, at least one, and preferably each, of R₁ and R₂ is alkyl, and the total number of carbon atoms in R₁ and R₂ is from 2 to 6, or from 2 to 4, and is, for example, 2, 3, or 4.

According to some embodiments of any of the embodiments described herein, R₁ and R₂ are each independently selected from hydrogen, methyl, ethyl, propyl and isopropyl.

In exemplary embodiments, R₁ and R₂ are each methyl; or R₁ is methyl and R₂ is ethyl; or R₁ is isopropyl and R₂ is hydrogen.

According to some embodiments of any of the embodiments described herein, R₃-R₈ are each hydrogen; such that the tetrahydropyrane ring in Formula I is substituted solely at the position adjacent to the oxygen (when one or both of R1 and R2 is/are an alkyl as described herein). Alternatively, the tetrahydropyrane ring is substituted at one or more other positions, such that one or more of R₃-R₈ is other than hydrogen.

According to some embodiments of any of the embodiments described herein, R₃-R₁₃ are each hydrogen.

According to some embodiments of any of the embodiments described herein, R₉-R₁₃ are each hydrogen.

According to some embodiments of any of the embodiments described herein, R₁₁-R₁₃ are each hydrogen.

According to some embodiments of any of the embodiments described herein, one or more of R₃-R₁₃ is other than hydrogen, and can be, for example, alkyl, cycloalkyl, halo, haloalkyl, hydroxy, alkoxy, thiol, thioalkoxy, amine, cyano and nitro, or any of the other substituents as described herein.

According to some embodiments of any of the embodiments described herein, R₁₁ and R₁₂ are each hydrogen, and at least one of R₁₃ and R₁₄ is other than hydrogen, and can be, for example, alkyl, cycloalkyl, halo, haloalkyl, hydroxy, alkoxy, thiol, thioalkoxy, amine, cyano and nitro, or any of the other substituents as described herein.

According to some embodiments of any of the embodiments described herein, one or more of R₃-R₁₃ is alkyl, preferably a lower alkyl, of from 1 to 6, or from 1 to 4 carbon atoms in length, preferably a lower unsubstituted alkyl, which can be linear or branched and is preferably linear.

According to some embodiments of any of the embodiments described herein, R₁₄ is alkyl, preferably a lower alkyl, of from 1 to 6, or from 1 to 4 carbon atoms in length, preferably a lower unsubstituted alkyl, which can be linear or branched and is preferably linear (e.g., methyl, ethyl, propyl). According to some of these embodiments, R₃-R₁₃ are each hydrogen.

According to some embodiments of any of the embodiments described herein, R₁₄ is methyl. According to some of these embodiments, R₃-R₁₃ are each hydrogen.

When R₁₄ is other than hydrogen, the carbon to which R₁₄ is attached features an asymmetric stereoconfiguration. Embodiments of the present invention encompass both the S- and R-stereoconfigurations.

According to some embodiments of any of the embodiments described herein, each of R₁₅-R₁₉ is hydrogen.

According to some embodiments of any of the embodiments described herein, at least one of R₁₅-R₁₉ is an electron-donating substituent, for example, fluoro, alkyl, cycloalkyl, hydroxy, alkoxy, aryloxy, amine, thiol, thioalkoxy, thioaryloxy, and like substituents. According to According to some embodiments, one or more of the substituents at the ortho and/or para position(s) with respect to the tetrahydropyrane substituent, that is, one or more of R₁₅, R₁₇ or R₁₉ is an electron donating substituent or is selected from fluoro, alkyl, cycloalkyl, hydroxy, amine, alkoxy, aryloxy, thiol, thioalkoxy, thioaryloxy, and like substituents.

According to some embodiments of any of the embodiments described herein, when at least one of R₁₅-R₁₉ is an electron-donating substituent, as described herein, the substituent is “aprotic”, such that it is other than hydroxy, thiol or primary amine, or is other than hydroxy, thiol, primary amine, or secondary amine.

According to some embodiments of any of the embodiments described herein, one or more of R₁₅, R₁₇ or R₁₉ is alkyl (preferably a lower alkyl as described herein), fluoro, thioalkoxy (preferably lower thioalkoxy) or alkoxy (preferably a lower alkoxy, or from 1 to 6, or from 1 to 4 carbon atoms in length).

By “lower alkoxy” or “lower thioalkoxy” it is meant that the alkyl in such a group is a lower alkyl as described herein in any of the respective embodiments and any combination thereof. In some embodiments, the alkyl is a linear alkyl. In some embodiments the alkyl is unsubstituted.

According to some embodiments of any of the embodiments described herein, R₁₅ is alkoxy (preferably lower alkoxy such as methoxy or ethoxy or propoxy or isopropoxy or butoxy). According to some embodiments of any of the embodiments described herein, R₁₅ is methoxy.

According to some embodiments of any of the embodiments described herein, each of R₂₀-R₂₄ is hydrogen.

According to some embodiments of any of the embodiments described herein, at least one of R₂₀-R₂₄ is an electron-donating substituent, for example, fluoro, alkyl, cycloalkyl, hydroxy, alkoxy, aryloxy, amine, thiol, thioalkoxy, thioaryloxy, and like substituents. According to According to some embodiments, one or more of the substituents at the ortho and/or para position(s) with respect to the attachment position, that is, one or more of R₂₀, R₂₂ or R₂₄ is an electron donating substituent or is selected from fluoro, alkyl, cycloalkyl, hydroxy, amine, alkoxy, aryloxy, thiol, thioalkoxy, thioaryloxy, and like substituents.

According to some embodiments of any of the embodiments described herein, when at least one of R₂₀-R₂₄ is an electron-donating substituent, as described herein, the substituent is “aprotic”, such that it is other than hydroxy, thiol or primary amine, or is other than hydroxy, thiol, primary amine, or secondary amine.

According to some embodiments of any of the embodiments described herein, one or more of R₂₀-R₂₄ or one or more of R₂₀, R₂₂ or R₂₄ is fluoro, amine (preferably a tertiary amine), or alkoxy (preferably a lower alkoxy, as described herein).

According to some embodiments of any of the embodiments described herein, R₂₂ is amine (preferably a tertiary amine), fluoro or alkoxy (preferably lower alkoxy).

According to some embodiments of any of the embodiments described herein, R₂₂ is amine (preferably a tertiary amine).

According to some embodiments of any of the embodiments described herein, the tertiary amine is preferably —NR′R″, with each of R′ and R″ being independently alkyl or cycloalkyl, preferably each is alkyl, which can be the same or different. In some embodiments, the alkyl is a lower alkyl as described herein, for example methyl.

According to some embodiments of any of the embodiments described herein, one or more of R₂₀-R₂₄ or one or more of R₂₀, R₂₂ or R₂₄, or R₂₂ is dialkyl amine, for example, dimethylamine.

According to some embodiments of any of the embodiments described herein, an exemplary compound of Formula I is:

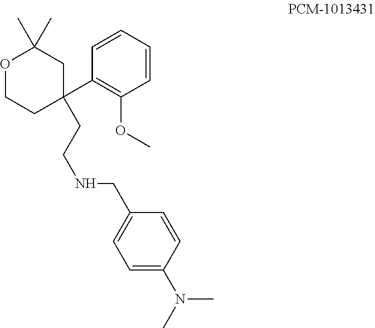 [SMILES: COc1ccccc1C1(CCNCc2ccc(N(C)C)cc2)CCOC(C)(C)C1]

(also referred to herein as C4).

According to some embodiments of any of the embodiments described herein, exemplary compounds of Formula I include:

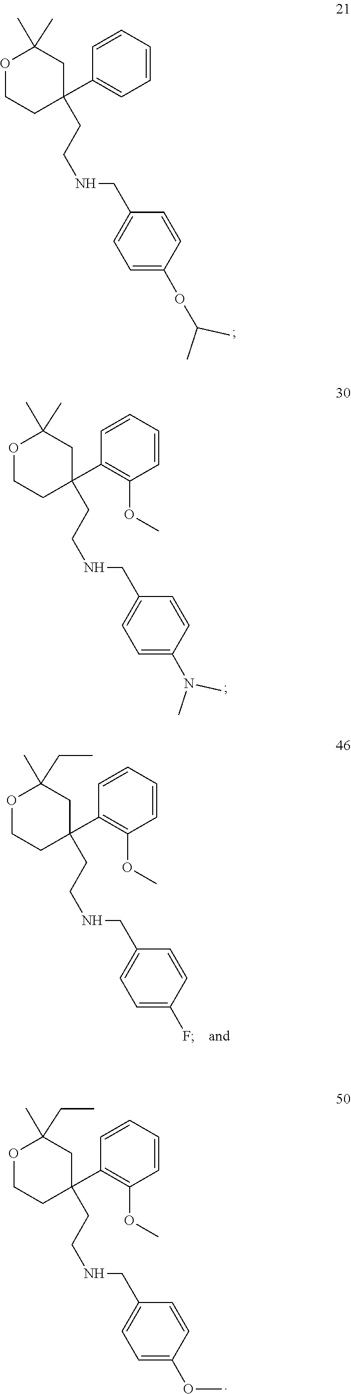 [SMILES: CC(C)Oc1ccc(CNCCC2(c3ccccc3)CCOC(C)(C)C2)cc1 ; COc1ccccc1C1(CCNCc2ccc(N(C)C)cc2)CCOC(C)(C)C1 ; CCC1(C)CC(CCNCc2ccc(F)cc2)(c2ccccc2OC)CCO1 ; "and" ; CCC1(C)CC(CCNCc2ccc(OC)cc2)(c2ccccc2OC)CCO1]

According to some embodiments of any of the embodiments described herein, According to some embodiments of any of the embodiments described herein, exemplary compounds of Formula I include:

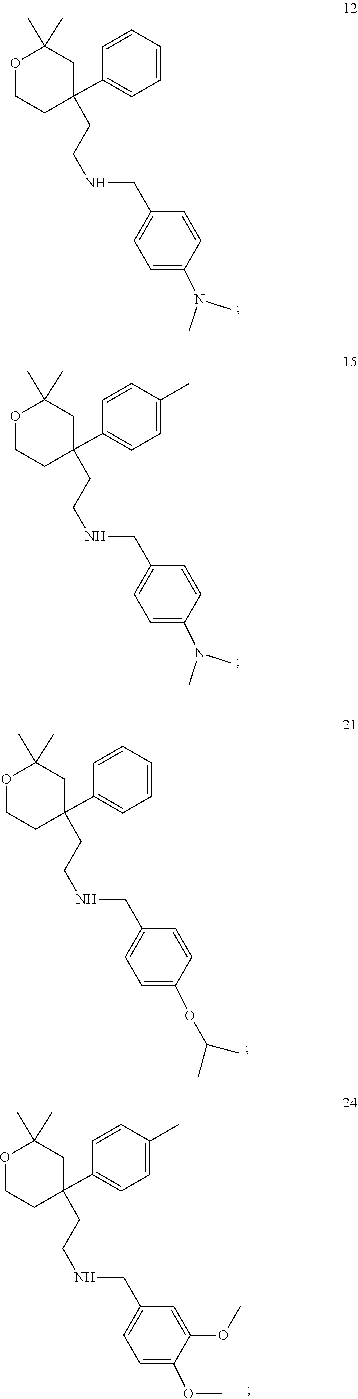 [SMILES: CN(C)c1ccc(CNCCC2(c3ccccc3)CCOC(C)(C)C2)cc1 ; Cc1ccc(C2(CCNCc3ccc(N(C)C)cc3)CCOC(C)(C)C2)cc1 ; CC(C)Oc1ccc(CNCCC2(c3ccccc3)CCOC(C)(C)C2)cc1 ; COc1ccc(CNCCC2(c3ccc(C)cc3)CCOC(C)(C)C2)cc1OC]

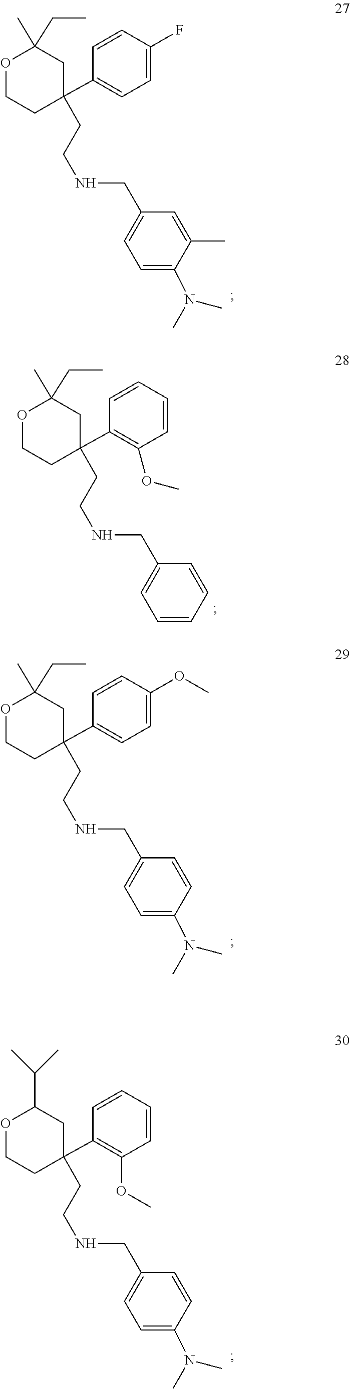 [SMILES: CCC1(C)CC(CCNCc2ccc(N(C)C)c(C)c2)(c2ccc(F)cc2)CCO1 ; CCC1(C)CC(CCNCc2ccccc2)(c2ccccc2OC)CCO1 ; CCC1(C)CC(CCNCc2ccc(N(C)C)cc2)(c2ccc(OC)cc2)CCO1 ; COc1ccccc1C1(CCNCc2ccc(N(C)C)cc2)CCOC(C(C)C)C1]

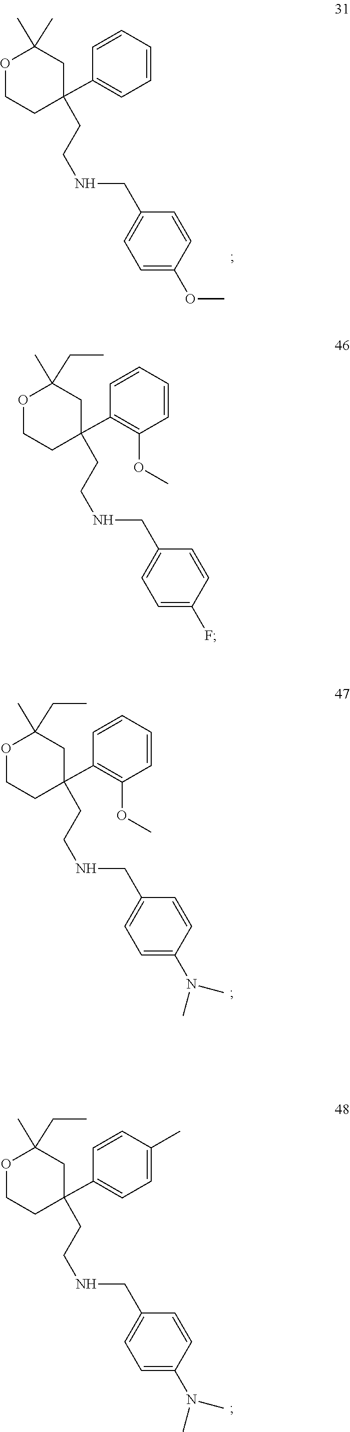 [SMILES: COc1ccc(CNCCC2(c3ccccc3)CCOC(C)(C)C2)cc1 ; CCC1(C)CC(CCNCc2ccc(F)cc2)(c2ccccc2OC)CCO1 ; CCC1(C)CC(CCNCc2ccc(N(C)C)cc2)(c2ccccc2OC)CCO1 ; CCC1(C)CC(CCNCc2ccc(N(C)C)cc2)(c2ccc(C)cc2)CCO1]

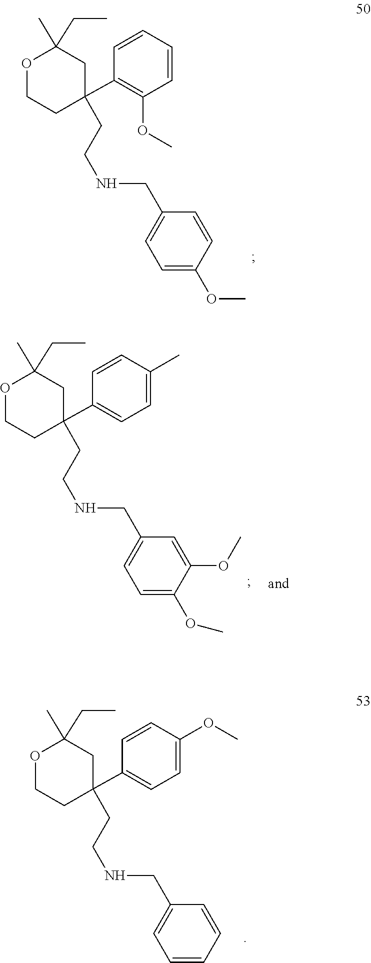 [SMILES: CCC1(C)CC(CCNCc2ccc(OC)cc2)(c2ccccc2OC)CCO1 ; CCC1(C)CC(CCNCc2ccc(OC)c(OC)c2)(c2ccc(C)cc2)CCO1 ; CCC1(C)CC(CCNCc2ccccc2)(c2ccc(OC)cc2)CCO1]

According to some embodiments of any of the embodiments described herein, the compound is represented by Formula II:

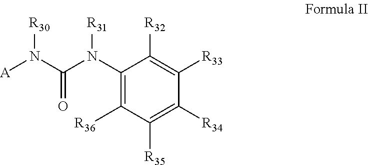

wherein:
R₃₀ and R₃₁ are each independently selected from hydrogen, alkyl and cycloalkyl;
R₃₂-R₃₆ are each independently selected from hydrogen, alkyl, cycloalkyl, halo, haloalkyl, hydroxy, alkoxy, thiol, thioalkoxy, amine, cyano and nitro, provided that at least one of R₃₂-R₃₆, preferably at least one of R₃₂, R₃₄ and R₃₆ is halo; and
A is selected from:

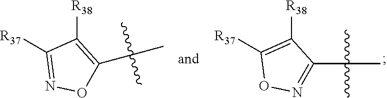

wherein the curved line represents an attachment point to NR₃₀₋; and R₃₇ and R₃₈ are each independently selected from hydrogen alkyl and cycloalkyl.

According to some embodiments of any of the embodiments described herein, R₃₀ and R₃₁ are each independently selected from hydrogen and alkyl.

According to some embodiments of any of the embodiments described herein, when one or each of R₃₀ and R₃₁ is alkyl, the alkyl is a lower alkyl as described herein.

According to some embodiments of any of the embodiments described herein, R₃₀ and R₃₁ are each hydrogen.

According to some embodiments of any of the embodiments described herein, one or more of R₃₂-R₃₆ is halo and the remaining variables are each hydrogen. Alternatively, one or more of the remaining variables is alkyl (preferably lower alkyl), cycloalkyl, haloalkyl, hydroxy, alkoxy, thiol, thioalkoxy, amine, cyano and nitro, preferably alkyl, or haloalkyl (e.g., CF₃).

According to some embodiments of any of the embodiments of Formula II, the one or more halo substituents can be chloro, bromo and/or iodo, or chloro and/or bromo, or chloro.

According to some embodiments of any of the embodiments of Formula II, one or more of R₃₂, R₃₄ and R₃₆ is halo, and according to some embodiments, one or more of R₃₂, R₃₄ and R₃₆ is chloro.

According to some embodiments of any of the embodiments of Formula II, one or more of R₃₂, R₃₄ and R₃₆ is halo (e.g., chloro), and each of R₃₀ and R₃₁ is hydrogen. According to some of these embodiments, one or more of R₃₂-R₃₆ which is not halo are each hydrogen.

According to some embodiments of any of the embodiments described herein, A is

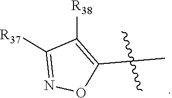

According to some of these embodiments, at least two of R₃₂-R₃₆, preferably at least two of R₃₂, R₃₄ and R₃₆, are each halo, preferably chloro. According to some of these embodiments, one or more of R₃₂-R₃₆ which is not halo are each hydrogen. According to some of these embodiments, each of R₃₀ and R₃₁ is hydrogen.

According to some embodiments of any of the embodiments described herein, A is

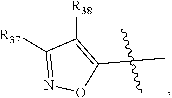

two of R₃₂, R₃₄ and R₃₆, are each halo, preferably chloro and the third is hydrogen, and R₃₃ and R₃₅ are each hydrogen. According to some of these embodiments, each of R₃₀ and R₃₁ is hydrogen.

According to some embodiments of any of the embodiments described herein, A is

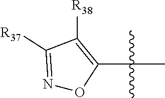

and no more than one of R₃₂-R₃₆, preferably one of R₃₂, R₃₄ and R₃₆, is halo, preferably chloro. According to some of these embodiments, one or more of R₃₂-R₃₆ which is not halo are each hydrogen. According to some of these embodiments, each of R₃₀ and R₃₁ is hydrogen.

According to some embodiments of any of the embodiments described herein, A is

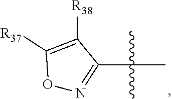

R₃₂ is halo, preferably chloro, and R₃₃-R₃₆ are each hydrogen. According to some of these embodiments, each of R₃₀ and R₃₁ is hydrogen.

According to some embodiments of any of the embodiments described herein, R₃₇ and R₃₈ are each independently selected from hydrogen and alkyl.

According to some of any of the embodiments described herein, A is

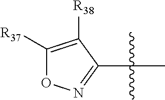

and one or more of R₃₇ and R₃₈ is alkyl, preferably a lower alkyl (e.g., methyl). According to some of these embodiments, R₃₇ is alkyl (e.g., methyl).

According to some of any of the embodiments described herein, A is

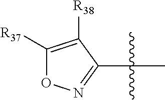

and R₃₇ and R₃₈ are each hydrogen.

It is to be noted that for any of the embodiments described herein for Formula II, A can be a heteroaryl other than isoxazole, and can be, for example, furane, pyrazole, triazole, diazole, oxadiazole, pyrrole, and other heteroaryls of 5, 6, 7-membered ring and one, two or more heteroatoms selected from nitrogen and oxygen.

According to some embodiments of any of the embodiments described herein, the compound is:

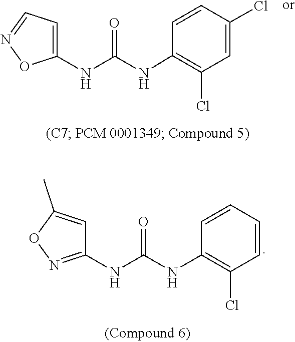 [SMILES: O=C(Nc1ccno1)Nc1ccc(Cl)cc1Cl ; "or" ; Cc1cc(NC(=O)Nc2ccccc2Cl)no1]

According to some embodiments of any of the embodiments described herein, the compound is represented by Formula III:

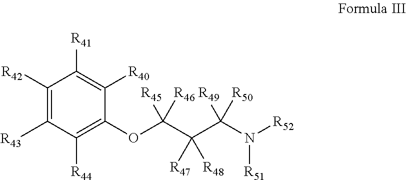

wherein:
R₄₀-R₄₄ are each independently selected from hydrogen, alkyl, cycloalkyl, halo, hydroxy, alkoxy, thiol, thioalkoxy, amine; and
R₄₅-R₅₂ are each independently selected from hydrogen, alkyl, cycloalkyl, halo, hydroxy, alkoxy, thiol, thioalkoxy, and amine.

According to some embodiments of any of the embodiments described herein, at least one of R₄₀-R₄₄, is other than hydrogen, and in some embodiments, one or more of R₄₀-R₄₄, is alkyl, cycloalkyl, halo, alkoxy, thioalkoxy, or amine (e.g., a tertiary amine).

According to some embodiments of any of the embodiments described herein, at least one of R₄₀-R₄₄ is cycloalkyl.

According to some embodiments of any of the embodiments described herein, one of R₄₀-R₄₄ is cycloalkyl.

According to some embodiments of any of the embodiments described herein, R₄₀ is other than hydrogen and is alkyl, cycloalkyl, halo, alkoxy, thioalkoxy, or amine (e.g., a tertiary amine).

According to some embodiments of any of the embodiments described herein, R₄₀ is cycloalkyl.

According to some embodiments of any of the embodiments described herein, the cycloalkyl is cyclopentyl, cyclobutyl, cyclopropyl, cyclohexyl, cycloheptyl or cyclooctyl.

According to some embodiments of any of the embodiments described herein, the cycloalkyl is cyclopentyl or cyclohexyl.

According to some embodiments of any of the embodiments described herein, R₄₀ is a cycloalkyl and is preferably cyclopentyl or cyclohexyl.

According to some embodiments of any of the embodiments described herein, when one or more of R₄₀-R₄₄ is other than hydrogen, the remaining variables can be each hydrogen, or, one or more can be other than hydrogen.

According to some embodiments of any of the embodiments described herein, R₄₀ is other than hydrogen (and is, for example, cycloalkyl) and R₄₁-R₄₄ are each hydrogen.

According to some embodiments of any of the embodiments described herein, R₄₀ is a cycloalkyl and at least one of R₄₁-R₄₄, is other than hydrogen, and in some embodiments, one or more of R₄₁-R₄₄, is alkyl, cycloalkyl, halo, alkoxy, thioalkoxy, or amine (e.g., a tertiary amine).

According to some embodiments of any of the embodiments described herein, each of R₄₅-R₅₀ is hydrogen.

According to some embodiments of any of the embodiments described herein, at least one of R₄₅-R₅₀ is other than hydrogen, and is selected from hydroxy, thiol, alkoxy and thioalkoxy.

According to some embodiments of any of the embodiments described herein, at least one of R₄₅-R₅₀ is hydroxy.

According to some embodiments of any of the embodiments described herein, R₄₇ is hydroxy. According to some of these embodiments, R₄₅, R₄₆ and R₄₈-R₅₀ are each hydrogen. Alternatively, one or more of R₄₅, R₄₆ and R₄₈-R₅₀ can be, for example, alkyl (preferably lower alkyl), halo, hydroxy, thiol, amine, alkoxy, thioalkoxy or any of the other substituents as described herein.

According to some embodiments of any of the embodiments described herein, R₄₇ is thiol. According to some of these embodiments, R₄₅, R₄₆ and R₄₈-R₅₀ are each hydrogen. Alternatively, one or more of R₄₅, R₄₆ and R₄₈-R₅₀ can be, for example, alkyl (preferably lower alkyl), halo, hydroxy, thiol, amine, alkoxy, thioalkoxy or any of the other substituents as described herein.

According to some embodiments of any of the embodiments described herein, R₄₅ is hydroxy or thiol. According to some of these embodiments, R₄₆-R₅₀ are each hydrogen. Alternatively, one or more of R₄₆-R₅₀ can be, for example, alkyl (preferably lower alkyl), halo, hydroxy, thiol, amine, alkoxy, thioalkoxy or any of the other substituents as described herein.

According to some embodiments of any of the embodiments described herein, R₅₀ is hydroxy or thiol. According to some of these embodiments, R₄₅-R₄₉ are each hydrogen. Alternatively, one or more of R₄₅-R₄₉ can be, for example, alkyl (preferably lower alkyl), halo, hydroxy, thiol, amine, alkoxy, thioalkoxy or any of the other substituents as described herein.

According to some embodiments of any of the embodiments described herein, each of R₅₁ and R₅₂ is hydrogen, such that the amine is a primary amine.

According to some embodiments of any of the embodiments described herein, one or each of R₅₁ and R₅₂ is other than hydrogen, such that the amine or secondary or tertiary amine.

According to some embodiments of any of the embodiments described herein, R₅₂ is other than hydrogen and in some embodiments it is an alkyl. In some of these embodiments, the alkyl is a lower alkyl, as described herein, which can be linear or branched, and is preferably unsubstituted. In some of these embodiments, the alkyl is a lower branched (e.g., bulky) alkyl, such as isopropyl, isobutyl, tert-butyl, isopentyl, amyl, etc. In exemplary embodiments, R₅₂ is tert-butyl. In some of any of these embodiments, R₅₁ is hydrogen.

According to some embodiments of any of the embodiments described herein, the compound is:

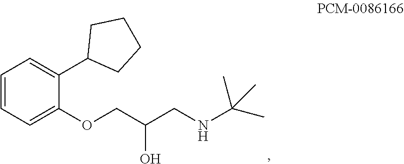 [SMILES: CC(C)(C)NCC(O)COc1ccccc1C1CCCC1]

also referred to herein as C2.

According to some embodiments of any of the embodiments described herein, the compound is represented by Formula IV:

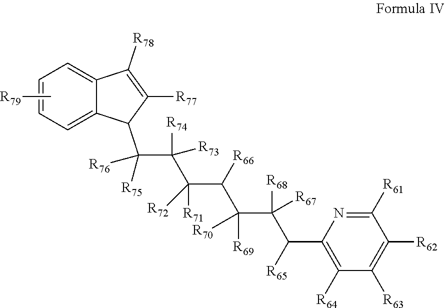

wherein:
R₆₁-R₆₄ are each independently selected from hydrogen, alkyl, cycloalkyl, halo, haloalkyl, amine, hydroxy, alkoxy, thiol, thioalkoxy, or, alternatively, two of R₆₁-R₆₄ form together a cyclic ring, which can be alicyclic, heteroalicyclic, aryl or heteroaryl, as these are defined herein;
R₆₅ and R₆₆ are each independently selected from hydrogen, alkyl and cycloalkyl;
R₆₇-R₇₈ are each independently selected from hydrogen, alkyl, cycloalkyl, halo, haloalkyl, amine, hydroxy, alkoxy, thiol and thioalkoxy; and
R₈₀ represents one or more (1, 2, 3, 4 or 5) substituents on the phenyl ring, or is absent, wherein each of these substituents, if present, is independently selected from alkyl, cycloalkyl, halo, haloalkyl, amine, hydroxy, alkoxy, thiol, and thioalkoxy.

According to some embodiments of any of the embodiments described herein, one or each of R₆₅ and R₆₆ is selected from hydrogen and alkyl.

According to some embodiments of any of the embodiments described herein, each of R₆₅ and R₆₆ is hydrogen.

According to some embodiments of any of the embodiments described herein, one or more of R₆₁-R₆₄ is other than hydrogen.

According to some embodiments of any of the embodiments described herein, one or more of R₆₁-R₆₄ is selected from alkyl, haloalkyl and halo, and is preferably a haloalkyl such as trihaloalkyl (e.g., CF₃).

According to some embodiments of any of the embodiments described herein, when one or more of R₆₁-R₆₄ is other than hydrogen, the others can be hydrogen or any of the other substituents.

According to some embodiments of any of the embodiments described herein, one or more of R₆₁-R₆₄ is selected from alkyl, haloalkyl and halo and is preferably a haloalkyl such as trihaloalkyl (e.g., CF₃), and the others can be hydrogen or any of the other substituents.

According to some embodiments of any of the embodiments described herein, R₆₄ is other than hydrogen.

According to some embodiments of any of the embodiments described herein, R₆₄ is selected from alkyl, haloalkyl and halo, and is preferably a haloalkyl such as trihaloalkyl (e.g., CF₃).

According to some embodiments of any of the embodiments described herein, R₆₄ is other than hydrogen and is alkyl, haloalkyl or halo, and is preferably trihaloalkyl as described herein, R₆₁-R₆₃ are each hydrogen, or one or more is other than hydrogen, and can be any of the other substituents as described herein.

According to some embodiments of any of the embodiments described herein, R₆₇-R₇₀ are each hydrogen. Alternatively, one or more of R₆₇-R₇₀ is other than hydrogen, and can be, for example, hydroxy, thiol, amine, halo, alkyl, alkoxy, thioalkoxy, haloalkyl, or any of the other substituents as described herein. According to some embodiments of any of the embodiments described herein, one or more of R₆₇-R₇₀ is/are hydroxy.

According to some embodiments of any of the embodiments described herein, R₇₁-R₇₆ are each hydrogen. Alternatively, one or more of R₇₁-R₇₆ is other than hydrogen, and can be, for example, hydroxy, thiol, amine, halo, alkyl, alkoxy, thioalkoxy, haloalkyl, or any of the other substituents as described herein. According to some embodiments of any of the embodiments described herein, one or more of R₇₁-R₇₆ is/are hydroxy.

According to some embodiments of any of the embodiments described herein, at least one of R₇₁-R₇₆, preferably R₇₄, is hydroxy.

According to some embodiments of any of the embodiments described herein, R₇₁-R₇₃, R₇₅ and R₇₆ are each hydrogen and R₇₄ is hydroxy.

According to some embodiments of any of the embodiments described herein, R₇₇ and R₇₈ are each hydrogen. Alternatively, one or more is alkyl, for example, a lower alkyl as described herein.

According to some embodiments of any of the embodiments described herein, R₇₉ is absent. Alternatively, the respective phenyl ring is substituted by one or more substituents, as indicated.

According to some embodiments of any of the embodiments described herein, the compound is:

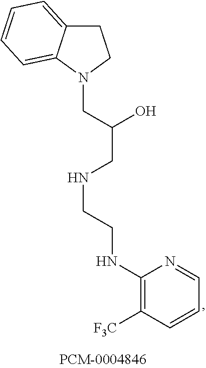 [SMILES: OC(CNCCNc1ncccc1C(F)(F)F)CN1CCc2ccccc21]

and is also referred to herein as C3.

According to some embodiments of any of the embodiments described herein, the compound is represented by Formula V:

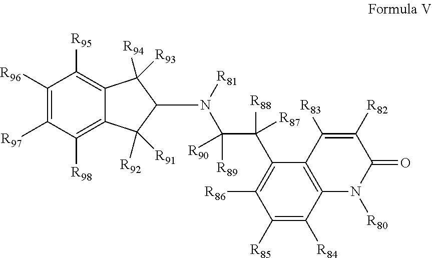

wherein:
R₈₀ and R₈₁ are each independently selected from hydrogen, alkyl and cycloalkyl;
R₈₂-R₈₆ are each independently selected from hydrogen, alkyl, cycloalkyl, halo, haloalkyl, amine, hydroxy, alkoxy, thiol, and thioalkoxy;
R₈₇-R₉₀ are each independently selected from hydrogen, alkyl, cycloalkyl, halo, haloalkyl, amine, hydroxy, alkoxy, thiol, and thioalkoxy;
R₉₁-R₉₄ are each independently selected from hydrogen, alkyl, cycloalkyl, halo, haloalkyl, amine, hydroxy, alkoxy, thiol, and thioalkoxy; and
R₉₅-R₉₈ are each independently selected from hydrogen, alkyl, cycloalkyl, halo, haloalkyl, amine, hydroxy, alkoxy, thiol, and thioalkoxy.

According to some embodiments of any of the embodiments described herein, R₈₀ and R₈₁ are each independently selected from alkyl and hydrogen, and according to some embodiments, each is hydrogen.

According to some embodiments of any of the embodiments described herein, R₈₂ and R₈₃ are each independently selected from alkyl (e.g., a lower alkyl) and hydrogen, and according to some embodiments, each is hydrogen.

According to some embodiments of any of the embodiments described herein, at least one of R₈₄-R₈₆ is other than hydrogen, and is some embodiments, at least one of R₈₄-R₈₆ is hydroxy, thiol, alkoxy, thioalkoxy or amine. According to some of these embodiments, at least one of R₈₄-R₈₆ is hydroxy. According to some of any of the embodiments described herein, R₈₄ is other than hydrogen, and in some embodiments it is hydroxy, thiol, alkoxy, thioalkoxy or amine. According to some of any of the embodiments described herein, R₈₄ is hydroxy. According to some of any of the embodiments described herein, when one or more of R₈₄-R₈₆ is other than hydrogen, the other variables can be hydrogen, or any other substituent, for example, alkyl, haloalkyl, halo, etc.

According to some of any of the embodiments described herein, R₈₄ is hydroxy and R₈₅ and R₈₆ are each hydrogen.

According to some embodiments of any of the embodiments described herein, each of R₈₇-R₉₀ is hydrogen.

According to some embodiments of any of the embodiments described herein, at least one of R₈₇-R₉₀ is other than hydrogen, and can be, for example, hydroxy, thiol, amine, thioalkoxy, and/or alkoxy. According to some of these embodiments, the remaining variables can be hydrogen, or, for example, alkyl, haloalkyl, halo and/or cycloalkyl.

According to some embodiments of any of the embodiments described herein, one or more of R₈₇-R₉₀ is hydroxy.

According to some embodiments of any of the embodiments described herein, R₈₇ is hydroxy, and R₈₈-R₉₀ are each hydrogen. Alternatively, one or more of R₈₈-R₉₀ is other than hydrogen and is a substituent as described herein.

According to some embodiments of any of the embodiments described herein, R₉₀ is hydroxy, and R₈₇-R₈₉ are each hydrogen. Alternatively, one or more of R₈₇-R₈₉ is other than hydrogen and is a substituent as described herein.

According to some embodiments of any of the embodiments described herein, each of R₉₁-R₉₄ is hydrogen. Alternatively, one or more of R₉₁-R₉₄ is other than hydrogen, and can be, independently, for example, alkyl, cycloalkyl, haloalkyl, halo, and like substituents.

According to some embodiments of any of the embodiments described herein, each of R₉₅-R₉₈ is hydrogen.

According to some embodiments of any of the embodiments described herein, one or more of R₉₅-R₉₈ is other than hydrogen and is independently selected from the substituents as described herein.

According to some embodiments of any of the embodiments described herein, one or more of R₉₅-R₉₈ is alkyl, preferably a lower alkyl. In some of these embodiments, the others are each hydrogen, or one or more can be a substituent other than hydrogen as described herein.

According to some embodiments of any of the embodiments described herein, two or more of R₉₅-R₉₈ is alkyl, preferably a lower alkyl. In some of these embodiments, the others are each hydrogen, or one or more can be a substituent other than hydrogen as described herein.

According to some embodiments of any of the embodiments described herein, one or more, or each, of R₉₆ and R₉₇ is an alkyl, preferably a lower alkyl. When both R₉₆ and R₉₇ is alkyl, it can be the same or different. According to some of these embodiments, the remaining R₉₅ and R₉₈ are each hydrogen.

According to some embodiments of any of the embodiments described herein, the compound is:

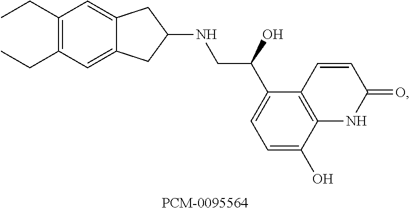 [SMILES: CCc1cc2c(cc1CC)CC(NC[C@@H](O)c1ccc(O)c3[nH]c(=O)ccc13)C2]

also referred to herein as C8.

According to some embodiments of any of the embodiments described herein, the compound is represented by Formula VI:

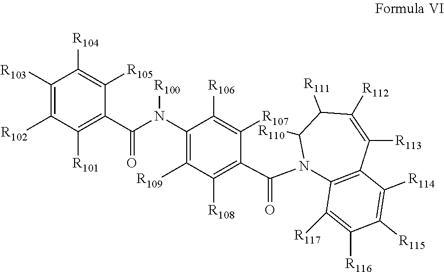

wherein:
R₁₀₀ is hydrogen, alkyl or cycloalkyl;
R₁₀₁-R₁₀₅ are each independently selected from hydrogen, alkyl, cycloalkyl, aryl, halo, haloalkyl, amine, hydroxy, alkoxy, thiol, thioalkoxy, heteroaryl, cyano, and nitro;
R₁₀₆-R₁₀₉ are each independently selected from hydrogen, alkyl, cycloalkyl, halo, haloalkyl, amine, hydroxy, alkoxy, thiol and thioalkoxy;
R₁₁₀-R₁₁₃ are each independently selected from hydrogen, alkyl, cycloalkyl, halo, haloalkyl, amine, hydroxy, alkoxy, thiol and thioalkoxy, or, alternatively, two of R₁₁₀-R₁₁₃ form together a substituted or unsubstituted cyclic ring (alicyclic, heteroalicyclic, aryl or heteroaryl); and
R₁₁₄-R₁₁₇ are each independently selected from hydrogen, alkyl, cycloalkyl, halo, haloalkyl, amine, hydroxy, alkoxy, thiol and thioalkoxy.

According to some embodiments of any of the embodiments described herein, R₁₀₀ is hydrogen or alkyl, and is preferably hydrogen.

According to some embodiments of any of the embodiments described herein, at least one of R₁₀₁-R₁₀₅ is other than hydrogen and can be, for example, alkyl, cycloalkyl, haloalkyl or aryl.

According to some embodiments of any of the embodiments described herein, at least one of R₁₀₁-R₁₀₅ is aryl, preferably phenyl.

According to some embodiments of any of the embodiments described herein, R₁₀₁ is other than hydrogen and can be, for example, alkyl, cycloalkyl, haloalkyl or aryl.

According to some embodiments of any of the embodiments described herein, R₁₀₁ is aryl According to some of these embodiments, R₁₀₂-R₁₀₅ are each hydrogen. (e.g., phenyl). Alternatively, one or more of R₁₀₂-R₁₀₅ is other than hydrogen and each independently can be a substituent as described herein.

According to some embodiments of any of the embodiments described herein, two of R₁₁₀-R₁₁₃ form together a substituted or unsubstituted cyclic ring, which can be alicyclic, aryl, heteroalicyclic or heteroaryl, as these terms are defined herein, wherein each can be substituted or substituted, as defined herein. According to some embodiments of any of the embodiments described herein, two of R₁₁₀-R₁₁₃ form together a substituted or unsubstituted heteroaryl, for example, a 5-membered, or six-membered heteroaryl.

According to some embodiments of any of the embodiments described herein, R₁₁₂ and R₁₁₃ form together a substituted or unsubstituted cyclic ring, which can be alicyclic, aryl, heteroalicyclic or heteroaryl, as these terms are defined herein, wherein each can be substituted or substituted, as defined herein. According to some embodiments of any of the embodiments described herein, R₁₁₂ and R₁₁₃ form together a substituted or unsubstituted heteroaryl, for example, a 5-membered, or six-membered heteroaryl.

According to some embodiments of any of the embodiments described herein, R₁₁₂ and R₁₁₃ form together a substituted or unsubstituted imidazole, for example, 2-methyl-2H-imidazole.

According to some embodiments of any of the embodiments described herein, R₁₁₀ and R₁₁₁ are each hydrogen. Alternatively, one or each of R₁₁₀ and R₁₁₁ can be other than hydrogen, as described herein, for example, can independently be alkyl, haloalkyl, and cycloalkyl, etc.

According to some embodiments of any of the embodiments described herein, R₁₀₆-R₁₀₉ are each hydrogen and/or R₁₁₄-R₁₁₇ are each hydrogen. Alternatively, one or more of these variables can be other than hydrogen, and can be, for example, alkyl, cycloalkyl, halo, haloalkyl, hydroxy, alkoxy, and/or any other substituent as described herein.

According to some embodiments of any of the embodiments described herein, the compound is:

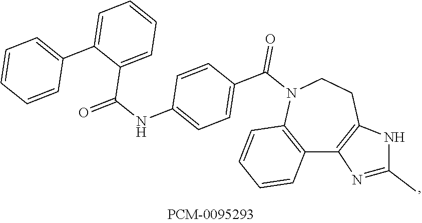 [SMILES: Cc1nc2c([nH]1)CCN(C(=O)c1ccc(NC(=O)c3ccccc3-c3ccccc3)cc1)c1ccccc1-2]

also referred to herein as C6.

According to some embodiments of any of the embodiments described herein, the compound is represented by Formula VII:

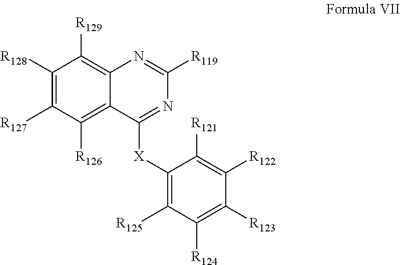

wherein:
X is O, S or NR₁₂₀;
R₁₁₉ and R₁₂₀ (if present) are each independently selected from hydrogen, alkyl or cycloalkyl;
R₁₂₁-R₁₂₅ are each independently selected from hydrogen, alkyl, cycloalkyl, aryl, halo, haloalkyl, amine, hydroxy, alkoxy, aryloxy, thioaryloxy, thiol, thioalkoxy, heteroaryl, cyano and nitro, or, alternatively or in addition, two of R₁₂₁-R₁₂₅ form together a substituted or unsubstituted cyclic ring (alicyclic, heteroalicyclic, aryl or heteroaryl); and
R₁₂₆-R₁₂₉ are each independently selected from hydrogen, alkyl, cycloalkyl, halo, haloalkyl, amine, hydroxy, alkoxy, thiol, thioalkoxy, heteroalicyclic, heteroaryl, and aryloxy.

According to some embodiments of any of the embodiments described herein, X is NR₁₂₀.

According to some embodiments of any of the embodiments described herein, R₁₁₉ and R₁₂₀, if present, are each independently selected from hydrogen and alkyl.

According to some embodiments of any of the embodiments described herein, R₁₁₉ and R₁₂₀, if present, are each hydrogen.

According to some embodiments of any of the embodiments described herein, at least one of R₁₂₁-R₁₂₅ is other than hydrogen.

According to some embodiments of any of the embodiments described herein, at least one of R₁₂₆-R₁₂₉ is other than hydrogen.

According to some embodiments of any of the embodiments described herein, at least one of R₁₂₁-R₁₂₅ and at least one of R₁₂₆-R₁₂₉ is other than hydrogen.

According to some embodiments of any of the embodiments described herein, at least one of R₁₂₁-R₁₂₅ is alkoxy or aryloxy.

According to some embodiments of any of the embodiments described herein, at least one of R₁₂₆-R₁₂₉ is alkoxy, aryloxy or a heterocyclic, for example, a heteroalicyclic or a heteroaryl.

According to some embodiments of any of the embodiments described herein, when one of R₁₂₆-R₁₂₉ is a heteroaryl, the remaining of these variables are each hydrogen.

According to some embodiments of any of the embodiments described herein, none of R₁₂₆-R₁₂₉ per se (alone) is a heteroaryl.

According to some embodiments of any of the embodiments described herein, none of R₁₂₆-R₁₂₉ per se (alone) is a heterocyclic ring, as described herein.

According to some embodiments of any of the embodiments described herein, two of R₁₂₁-R₁₂₅ form together a cyclic ring, which is preferably an alicyclic ring or a heteroalicyclic ring.

According to some embodiments of any of the embodiments described herein, two of R₁₂₁-R₁₂₅ form together a heteroalicyclic ring. The heteroalicyclic ring can be, for example, a 5-membered or 6-membered ring. The heteroalicyclic ring can include one or more heteroatoms, for example, one or more of O, N and S. In some embodiments, the heteroalicyclic ring comprises one or two oxygen atoms, and can be, for example, tetrahydrofurane, tetrahydropyrane, dioxolane, dioxane, and like cyclic moieties.

According to some embodiments of any of the embodiments described herein, R₁₂₁ and R₁₂₂ form together a cyclic ring as described herein in any of the respective embodiments, for example, dioxolane.

According to some embodiments of any of the embodiments described herein, two of R₁₂₁-R₁₂₅ form together a cyclic ring, the remaining variables can each be hydrogen or independently one or more of the other substituents as described herein, for example, alkyl, halo, haloalkyl, etc.

According to some embodiments of any of the embodiments described herein, at least two of R₁₂₆-R₁₂₉ are each independently an alkoxy.

According to some embodiments of any of the embodiments described herein, at least one, and preferably at least two, of R₁₂₆-R₁₂₉ are each independently an alkoxy, a thioalkoxy, thiol or hydroxy. In some of these embodiments, the remaining variables are each hydrogen, or, alternatively, one or more is other than hydrogen and can be a substituent as described herein.

According to some embodiments of any of the embodiments described herein, at least one, and preferably at least two, of R₁₂₆-R₁₂₉ are each independently an alkoxy.

According to some embodiments of any of the embodiments described herein, one or each of R₁₂₆ and R₁₂₈ is independently an alkoxy, a thioalkoxy, thiol or hydroxy. In some of these embodiments, the remaining variables are each hydrogen, or, alternatively, one or more is other than hydrogen and can be a substituent as described herein.

According to some embodiments of any of the embodiments described herein, one or each of R₁₂₆ and R₁₂₈ are each independently an alkoxy, and the compound is represented by Formula VIIa:

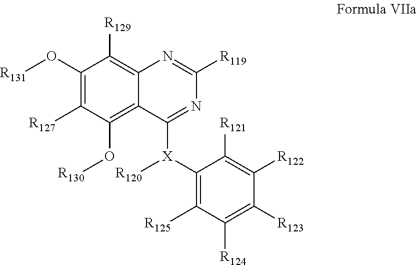

wherein R₁₃₀ and R₁₃₁ are each independently selected from alkyl, cycloalkyl, and heteroalicyclic and the other variables are as defined herein in any of the respective embodiments of Formula VII and any combination thereof.

According to some embodiments of any of the embodiments described herein for Formula VIIa, R₁₂₁ and R₁₂₂ form together the cyclic ring, preferably the heteroalicyclic ring, as described herein in any of the respective embodiments and any combination thereof.

According to some embodiments of any of the embodiments described herein for Formula VIIa, R₁₂₁ and R₁₂₂ form together a dioxolane.

According to some embodiments of any of the embodiments described herein, at least one of R₁₂₃-R₁₂₅, preferably R₁₂₅, is halo, preferably chloro.

According to some embodiments of any of the embodiments described herein, R₁₃₀ and R₁₃₁ each independently is or comprise a heteroalicyclic.

According to some embodiments of any of the embodiments described herein, R₁₃₀ is a heteroalicyclic, as defined herein, which can be, for example, a 5-membered or 6-membered ring comprising one or more of O, S and N. In some embodiments, R₁₃₀ is a 5-membered or 6-membered ring comprising one or more O atoms, and can be, for example, tetrahydrofurane, tetrahydropyrane, dioxolane, and like cyclic moieties.

According to some embodiments of any of the embodiments described herein, R₁₃₀ is tetrahydropyrane.

According to some embodiments of any of the embodiments described herein, R₁₃₁ is an alkyl substituted by a heteroalicylic. In some of these embodiments, the alkyl is a lower alkyl, as described herein, for example, methylene, ethylene or propylene, which is substituted by or terminated by a heteroalicyclic. In some of these embodiments, the heteroalicyclic is a 5-membered or 6-membered ring comprising one or more of O, S and N. In some embodiments, R₁₃₀ is a 5-membered or 6-membered ring comprising one or more N atoms, and can be, for example, piperidine, piperazine, oxazolidine, imidazolidine, dihydropyrimidine, and like moieties. In exemplary embodiments, the heteroalicyclic ring is piperazine. According to some embodiments of any of the embodiments described herein, R₁₃₁ is ethylpiperazine.

According to some embodiments of any of the embodiments described herein, the compound is:

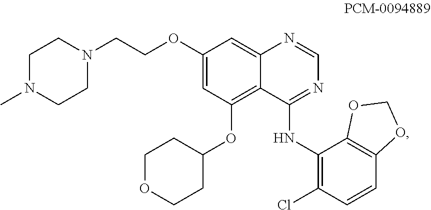 [SMILES: CN1CCN(CCOc2cc(OC3CCOCC3)c3c(Nc4c(Cl)ccc5c4OCO5)ncnc3c2)CC1]

and is also referred to herein as C1.

According to some embodiments of any of the embodiments described herein for Formula VIIa, R₁₃₀ is a heteroalicyclic and R₁₃₁ is an alkyl, as described herein in any of the respective embodiments, substituted or terminated by a heteroaryl.

According to some embodiments of any of the embodiments described herein for Formula VII, at least one of R₁₂₁-R₁₂₅ is aryloxy, preferably heteroaryloxy. According to some of these embodiments, the remaining variables can be each hydrogen, or can be independently a substituent as described herein.

According to some embodiments of any of the embodiments described herein, R₁₂₃ is the aryloxy, for example, a heteroaryloxy. According to some of these embodiments, R₁₂₁, R₁₂₂, R₁₂₄ and R₁₂₅ are each hydrogen. According to some of these embodiments, one or more of R₁₂₁, R₁₂₂, R₁₂₄ and R₁₂₅ is other than hydrogen and can be, for example, any of the substituents as described herein, for example, alkyl (e.g., lower alkyl).

According to some embodiments of any of the embodiments described herein for Formula VII, at least one of R₁₂₆-R₁₂₉ is a heteroaryl, which can be, for example, a 5-membered or 6-membered ring that comprises one or more O, N and/or S atoms. In some embodiments, at least one of R₁₂₆-R₁₂₉ is a 5-membered or 6-membered ring that comprises one or more O atoms, for example, furyl or pyranyl. In some embodiments, at least one of R₁₂₆-R₁₂₉ is furyl, more preferably a substituted furyl.

According to some embodiments of any of the embodiments described herein for Formula VII, R₁₂₇ is the heteroaryl.

According to some embodiments of any of the embodiments described herein for Formula VII, R₁₂₇ is furyl, and the compound is represented by Formula VIIb:

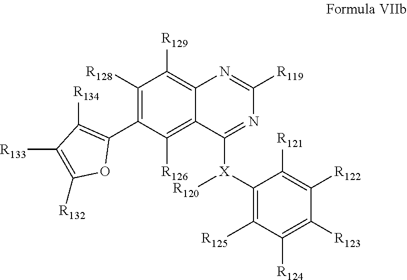

wherein R₁₃₂-R₁₃₄ are each independently selected from hydrogen, alkyl, cycloalkyl, halo, haloalkyl, amine, heteroalicyclic, heteroaryl, cyano and nitro, and the other variables are described herein for Formula VII, in any of the respective embodiments and any combination thereof.

According to some embodiments of any of the embodiments described herein for Formula VII, at least one of R₁₂₁-R₁₂₅ is an aryloxy, preferably a heteroaryloxy, as described herein in any of the respective embodiments.

According to some embodiments of any of the embodiments described herein for Formula VII, R₁₂₃ is a heteroaryloxy, preferably 7-triazolopyridinoxy. According to some of these embodiments, each of R₁₂₁, R₁₂₂, R₁₂₄ and R₁₂₅ is hydrogen. Alternatively, at least one of R₁₂₁, R₁₂₂, R₁₂₄ and R₁₂₅, preferably R₁₂₄, is alkyl, preferably methyl.

According to some embodiments of any of the embodiments described herein for Formula VIIb, at least one of R₁₃₂-R₁₃₄, preferably R₁₃₂, is an alkyl, preferably a substituted alkyl.

According to some embodiments of any of the embodiments described herein, the alkyl is substituted by a moiety that comprises an amine and/or SO₂, preferably a hydrocarbon interrupted and/or substituted by an amine and/or SO₂.

According to some embodiments of any of the embodiments described herein for Formula VIIb, at least one of R₁₃₂-R₁₃₄, preferably R₁₃₂, is an alkyl substituted by amine. According to some of these embodiments, the amine is substituted, that is, it is a secondary or tertiary amine. According to some of these embodiments, the amine is substituted by at least one alkyl which is further substituted by a sulfone.

According to some embodiments of any of the embodiments described herein for Formula VIIb, at least one of R₁₃₂-R₁₃₄, preferably R₁₃₂, is an alkyl and the alkyl is —CH₂—NH—CH₂—CH₂—SO₂—CH₃.

According to some embodiments of any of the embodiments described herein, the compound is:

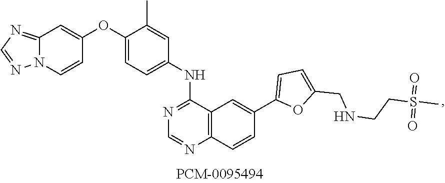 [SMILES: Cc1cc(Nc2ncnc3ccc(-c4ccc(CNCCS(C)(=O)=O)o4)cc23)ccc1Oc1ccn2ncnc2c1]

also referred to herein as C5.

According to some embodiments of any of the embodiments described herein for Formula VIIb, R₁₂₃ is other than heteroaryloxy; and/or R₁₃₂ is other than an alkyl substituted by a moiety that comprises an amine and/or SO₂, or a hydrocarbon interrupted and/or substituted by an amine and/or SO₂.

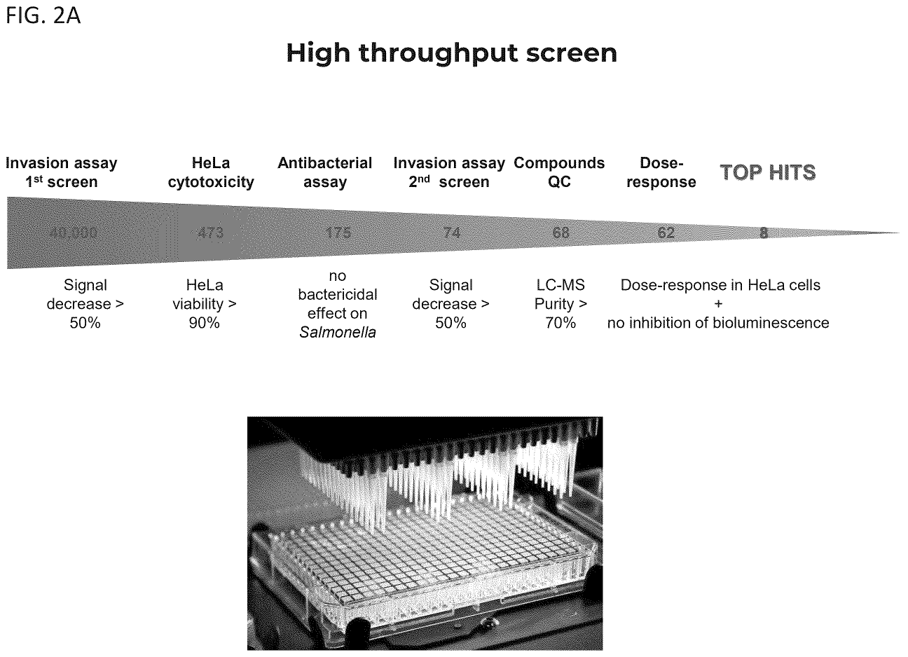

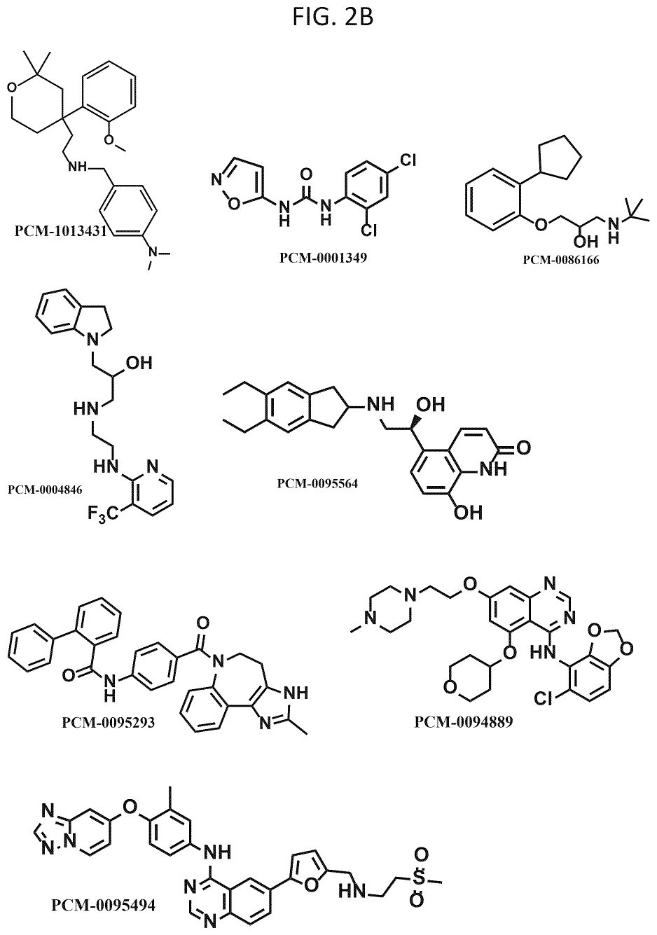

According to some embodiments of any of the embodiments described herein, the compound is one or more of the compounds presented in FIG. 2B.

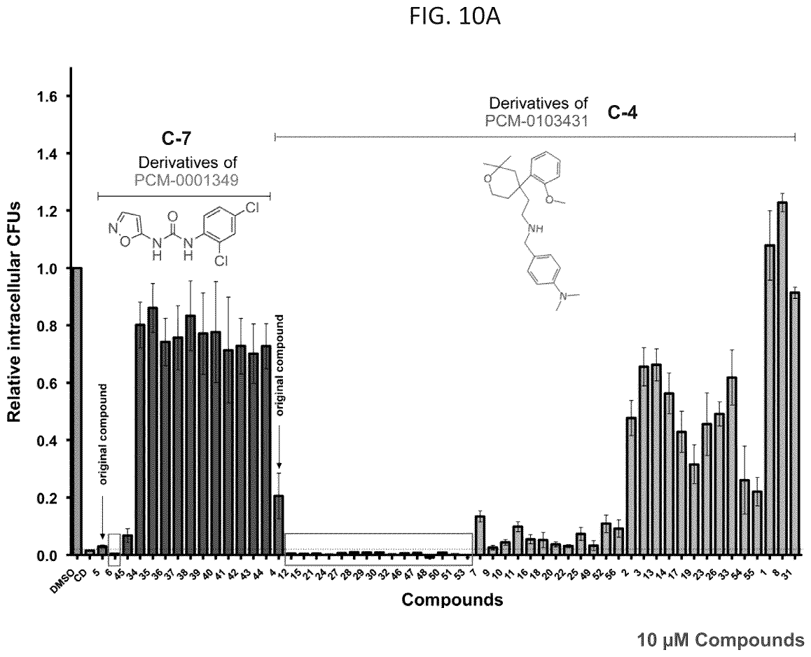

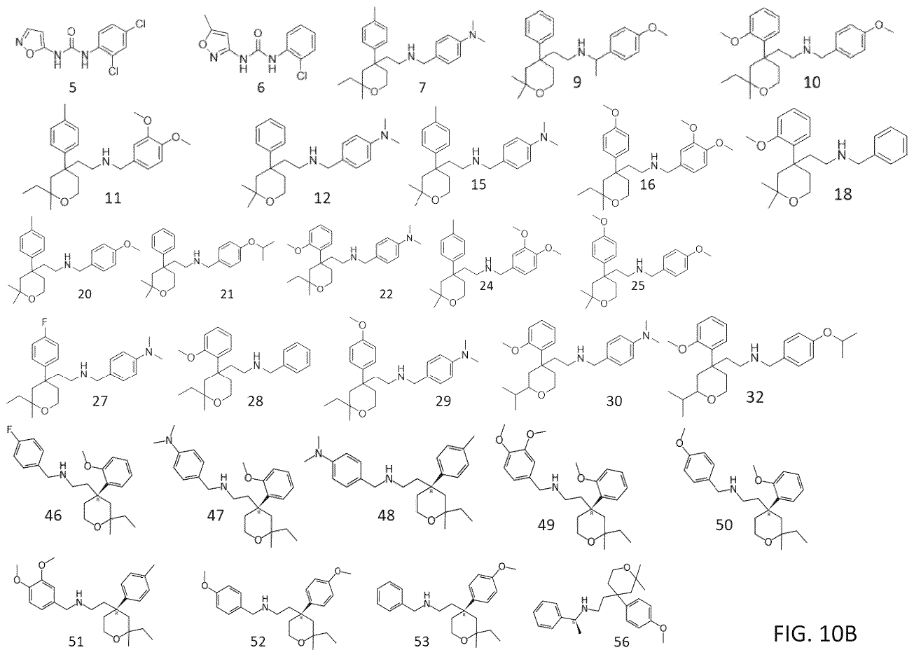

According to some embodiments of any of the embodiments described herein, the compound is one or more of the compounds presented in FIG. 10B.

Herein throughout, the phrase “linking moiety” or “linking group” describes a group that connects two or more moieties or groups in a compound. A linking moiety is typically derived from a bi- or tri-functional compound, and can be regarded as a bi- or tri-radical moiety, which is connected to two or three other moieties, via two or three atoms thereof, respectively.

Exemplary linking moieties include a hydrocarbon moiety or chain, optionally interrupted by one or more heteroatoms, as defined herein, and/or any of the chemical groups listed below, when defined as linking groups.

When a chemical group is referred to herein as “end group” it is to be interpreted as a substituent, which is connected to another group via one atom thereof.

The term “alkyl”, as used herein, describes a saturated aliphatic hydrocarbon including straight chain and branched chain groups. In some embodiments, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. In some embodiments, the alkyl is a lower alkyl having 1 to 8, or 1 to 6, or 1 to 4 carbon atoms. The alkyl group may be substituted or unsubstituted, as indicated herein.

The alkyl group can be an end group, as this phrase is defined hereinabove, wherein it is attached to a single adjacent atom, or a linking group, as this phrase is defined hereinabove, which connects two or more moieties via at least two carbons in its chain. When the alkyl is a linking group, it is also referred to herein as “alkylene” or “alkylene chain”.

The term “alkaryl” describes an alkyl, as defined herein, which is substituted by one or more aryl or heteroaryl groups. An example of alkaryl is benzyl.

Herein throughout, the term “alkyl” encompasses “alkaryl” unless specifically indicated otherwise.

The term alkenyl, as used herein, describes an alkyl, as defined herein, which contains a carbon-to-carbon double bond.

The term alkynyl, as used herein, describes an alkyl, as defined herein, which contains carbon-to-carbon triple bond.

The term “cycloalkyl” or “alicyclic” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The aryl group can be an end group, as this phrase is defined hereinabove, wherein it is attached to a single adjacent atom, or a linking group, as this phrase is defined hereinabove, which connects two or more moieties via at least two carbons in its chain. When the alkyl is a linking group, it is also referred to herein as “arylene”, for example, phenylene.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, carbazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyrane, morpholino and the like.

The term “alkoxy” describes both an —O-alkyl, an —O-cycloalkyl and an —O-heteroalicyclic group, as defined herein.

The term “aryloxy” describes an —O-aryl and an —O-heteroaryl, as defined herein.

The term “heteroaryloxy” describes specifically —O-heteroaryl.

Each of the alkyl, cycloalkyl, heteroalicyclic, aryl and heteroaryl groups, including alkylene and arylene groups, in the general formulas herein, including when included in alkoxy, thioalkoxy, thioaryloxy, aryloxy, etc., may be substituted by one or more substituents, whereby each substituent group can independently be, for example, halogen, alkyl, alkoxy, cycloalkyl, alkoxy, cyano, nitro, amine, hydroxyl, thiol, thioalkoxy, thiohydroxy, carboxy, amide, aryl and aryloxy, depending on the substituted group and its position in the molecule.

Additional substituents are also contemplated, for example, sulfinyl, sulfonyl, sulfonate, sulfate, azide, phosphonyl, phosphinyl, imine, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide.

The term “halide”, “halogen” or “halo” describes fluorine, chlorine, bromine or iodine.

The term “haloalkyl” describes an alkyl group as defined herein, further substituted by one or more halide(s). The term “haloalkyl” is encompassed by the term “alkyl”. The term “trihaloalkyl” describes an alkyl, typically methyl, which is substituted by 3 halo atoms, which can be the same or different. Examples include CF₃ and CCl₃, although other trihaloalkyls are contemplated.

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

Herein, the terms “amine” and “amino” each refer to either a —NR′R″ group or a —N⁺R′R″R″′ group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is not substituted by oxo (unless explicitly indicated otherwise), such that R′, R″ and R″′ are not (for example) carbonyl, C-carboxy or amide, as these groups are defined herein.

An “azide” group refers to a —N═N⁺═N⁻ group.

The term “carboxy” or “carboxylate” describes a —C(═O)—OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” describes a —C(═O)—R′ group, where R′ is as defined hereinabove.

The above-terms also encompass thio-derivatives thereof (thiocarboxy and thiocarbonyl).

The term “thiocarbonyl” describes a —C(═S)—R′ group, where R′ is as defined hereinabove.

A “thiocarboxy” group describes a —C(═S)—OR′ group, where R′ is as defined herein.

A “sulfinyl” group describes an —S(═O)—R′ group, where R′ is as defined herein.

A “sulfonyl” group describes an —S(═O)₂—R′ group, where Rx is as defined herein.

A “carbamyl” group describes an —OC(═O)—NR′R″ group, where R′ is as defined herein and R″ is as defined for R′.

A “nitro” group refers to a —NO₂ group.

A “cyano” or “nitrile” group refers to a —C≡N group.

Representative examples of nitrogen-containing heteroaryls include, but are not limited to thiadiazole, pyridine, pyrrole, oxazole, indole, purine and the like. Other moieties are also contemplated.

Representative examples of nitrogen-containing heteroalicyclic include, but are not limited to, morpholine, thiomorpholine, piperidine, piperazine, hexahydroazepine and tetrahydropyrane. Other moieties are also contemplated.

The term “piperazine” refers to a

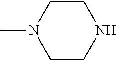

group or a

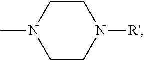

or a

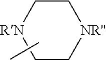

group, where R′ and R″ are as defined hereinabove.

The term “piperidine” refers to a

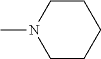

group or a

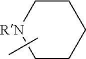

group, with R′ as defined herein.

The term “pyrrolidine” refers to a

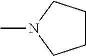

group or a

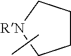

group, with R′ as defined herein.

The term “pyridine” refers to a

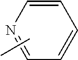

group.

The term pyrrole refers to a

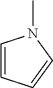

group or a

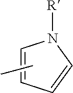

group, with R′ as defined herein.

The term “morpholine” refers to a

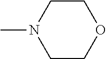

group, and encompasses also thiomorpholine.

The term “thiomorpholine” refers to a

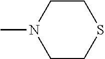

group.

The term “hexahydroazepine” refers to a

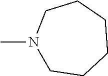

group.

A “C-carboxy” group refers to a —C(═O)—O—R′ group, where R′ is as defined herein.

An “O-carboxy” group refers to an R′C(═O)—O— group, where R′ is as defined herein.

A “carboxylic acid” group refers to a —C(═O)OH group.

An “oxo” group refers to a ═O group.

An “imine” group refers to a ═N—R′ group, where R′ is as defined herein.

An “oxime” group refers to a ═N—OH group.

A “hydrazone” group refers to a ═N—NR′R″ group, where each of R′ and R″ is as defined herein.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R′S(═O)₂—NR″-group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″-group, where each of R′ and R″ is as defined herein.

An “O-thiocarbamyl” group refers to an —OC(═S)—NR′R″ group, where each of R′ and R″ is as defined herein.

An “N-thiocarbamyl” group refers to an R′OC(═S)NR″-group, where each of R′ and R″ is as defined herein.

An “S-thiocarbamyl” group refers to an —SC(═O)—NR′R″ group, where each of R′ and R″ is as defined herein.

An “amide” or “amido” group encompasses C-amido and N-amido groups, as defined herein.

A “C-amido” group refers to a —C(═O)—NR′R″ group, where each of R′ and R″ is as defined herein.

An “N-amido” group refers to an R′C(═O)—NR″— group, where each of R′ and R″ is as defined herein.

A “urea group” refers to an —N(R′)—C(═O)—NR″R′″ group, where each of R′, R″ and R″ is as defined herein.

A “thiourea group” refers to a —N(R′)—C(═S)—NR″R′″ group, where each of R′, R″ and R″ is as defined herein.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a —PR′R″ group, with each of R′ and R″ as defined hereinabove.

The term “hydrazine” describes a —NR′—NR″R′″ group, with R′, R″, and R′″ as defined herein.

As used herein, the term “hydrazide” describes a —C(═O)—NR′—NR″R′″ group, where R′, R″ and R′″ are as defined herein.

As used herein, the term “thiohydrazide” describes a —C(═S)—NR′—NR″R′″ group, where R′, R″ and R″ are as defined herein.

A “guanidinyl” group refers to an —RaNC(═NRd)-NRbRc group, where each of Ra, Rb, Rc and Rd can be as defined herein for R′ and R″.

A “guanyl” or “guanine” group refers to an RaRbNC(═NRd)-group, where Ra, Rb and Rd are as defined herein.

For any of the embodiments described herein, the compound described herein may be in a form of a salt, for example, a pharmaceutically acceptable salt, and/or in a form of a prodrug.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” refers to a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

The compounds and structures described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### High-Throughput Screening (HTS) for Compounds Having Activity Against Intracellular Pathogens According to Some Embodiments of the Invention

To identify bioactive molecules that inhibit host cell infection by intracellular pathogens epithelial cells (HeLa) infection by the facultative intracellular pathogen Salmonella enterica serovar Typhimurium (S. Typhimurium) was used as a model. To achieve high specificity and a broad dynamic rage a reporter system was engineered that included the bacterial bioluminescence operon (luxCDABE) under the regulation of the S. Typhimurium gene sseK3. This gene is induced when Salmonella is intracellular, but suppressed when Salmonella is extracellular. This construct was introduced into S. Typhimurium strain SL1344 and used as the reporter strain during the screen in a way that the intensity of the bioluminescence signal is proportionate to the amount of intracellular bacteria. In other words, the more Salmonella invades, the higher the intensity of bioluminescence. The screen was performed in sterile tissue culture-treated white 384 flat bottom well plates (Greiner, #781080). 8×10³ HeLa cells were seeded in each well and incubated at 37 °C in 5% CO₂ humidified incubator for 4 h to let the cells adhere. Overall, 39,118 small molecules that were obtained from diverse bioactive compound libraries were added to the wells at a final concentration of 10 μM using the Multidrop Combi Reagent Dispenser (Thermo-Fisher Scientific).

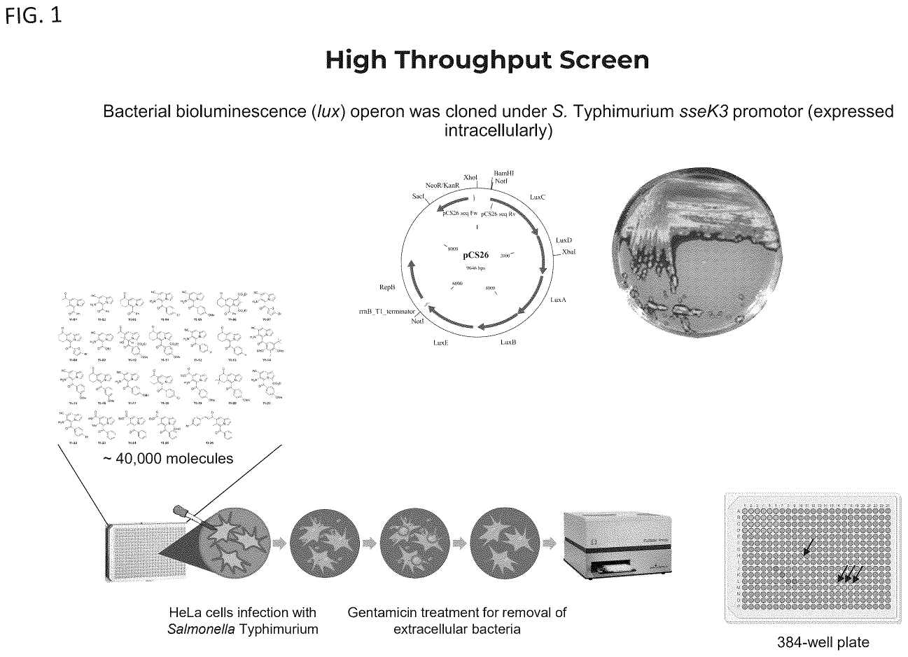

At the same time, an overnight culture of S. Typhimurium expressing the lux reporter system was subcultured 1:100 in LB medium and grown for 3 h at 37 °C with shaking (250 RPM) to the late logarithmic phase (OD₆₀₀~1). The cultures were then diluted 1:50 and aliquots of 25 μl were used to infect the cells at multiplicity of infection (MOI) of 1:50 (cell to bacteria). One h post-infection (p.i.), gentamicin at a final concertation of 20 μg/ml was added to each well and the plates were incubated for overnight (~16 h) at 37 °C in 5% CO₂ atmosphere. The next day, the infected cells were washed with phosphate-buffered saline (PBS) to remove all extracellular bacteria and the bioluminescence was detected by OMEGA-BMG Labtech, Ortenberg, Germany, Gain 3700, rapid plate reader to evaluate the amount of intracellular bacteria (FIG. 1).

### Example 2

### The Pipeline and the Screening Stages Used for the Identification of the Top Candidates in the HTS

Infection assay of the intracellular pathogen S. Typhimurium carrying the reporter system was performed in Hela cells in the presence of ~40,000 bioactive molecules. Compounds that were found to reduce the bioluminescence signal (N=473) were selected for the next stage. To filter out compounds that are cytotoxic to HeLa cells, the compounds examined for cytotoxicity in HeLa cells at 10 μM concentration. 175 compounds which presented less than 10% reduction in cell viability were selected for the next stage. In stage 3 the present inventors addressed the bactericidal effect of the compounds and filtered out 101 compounds that were bactericidal against S. Typhimurium. At stage 4, 74 compounds were tested again to confirm their ability to inhibit Salmonella infection. 68 hits that presented at least 50% decrease in the bioluminescent signal were selected for the next stage. 6/68 compounds that were found to be unstable, following liquid chromatography-mass spectrometry (LC-MS) or presented purity lower than 70% were excluded from the hit collection and the remaining 62 compounds were tested for their ability to inhibit Salmonella infection in dose-response infection experiments. At the end of the screen, 8 top hits that were able to inhibit salmonella infection in Hela cells, showed low cytotoxicity, no bactericidal activity and high purity, were identified (FIGS. 2A-B).

### Example 3

### The Inhibitory Effect of the Identified Compounds on Salmonella Infection

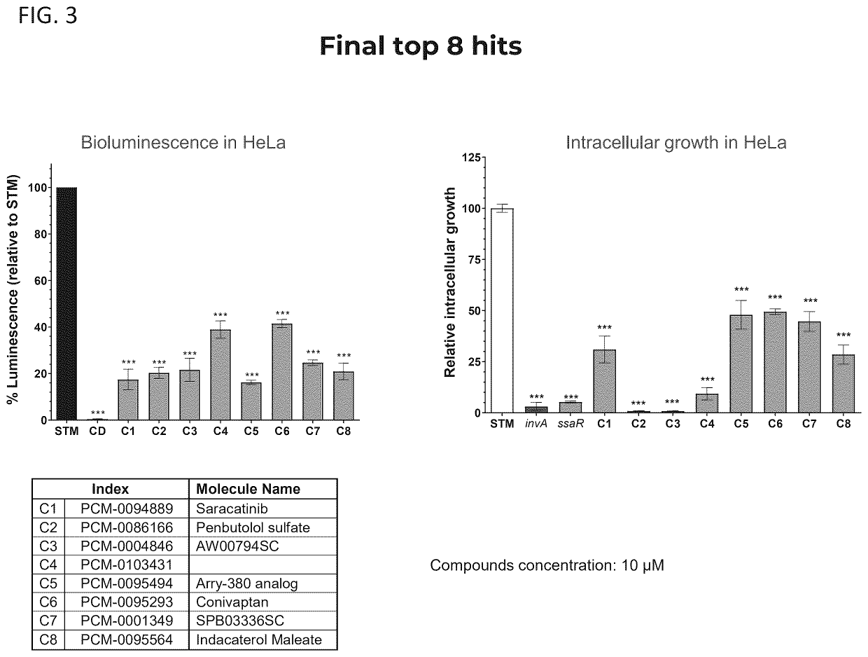

The ability of the top 8 hits to inhibit Salmonella Typhimurium (STM) invasion into HeLa cells was determined by the bioluminescence assay as described above (FIG. 3 left panel). Salmonella infection in the presence of the 8 compounds at a final concentration of 10 μM is shown relative to its infection in the absence of the compounds. Cytochalasin D (CD) that blocks actin polymerization and Salmonella invasion was used as a positive control. (FIG. 3 right panel) the effect of the compounds on Salmonella infection was tested independently by the gentamycin protection assay in the presence and absence of the compounds. Host cells infection by S. Typhimurium (STM) in the absence of compounds was used as a reference. STM isogenic mutants invA and ssaR that are impaired in their ability to invade and replicate in host cells, respectively were used as genetic negative controls.

FIG. 3 shows using two independent approaches that all eight top hits can effectively inhibit epithelial cells infection by S. Typhimurium.

### Example 4

### Cytotoxicity Testing of Identified Hits

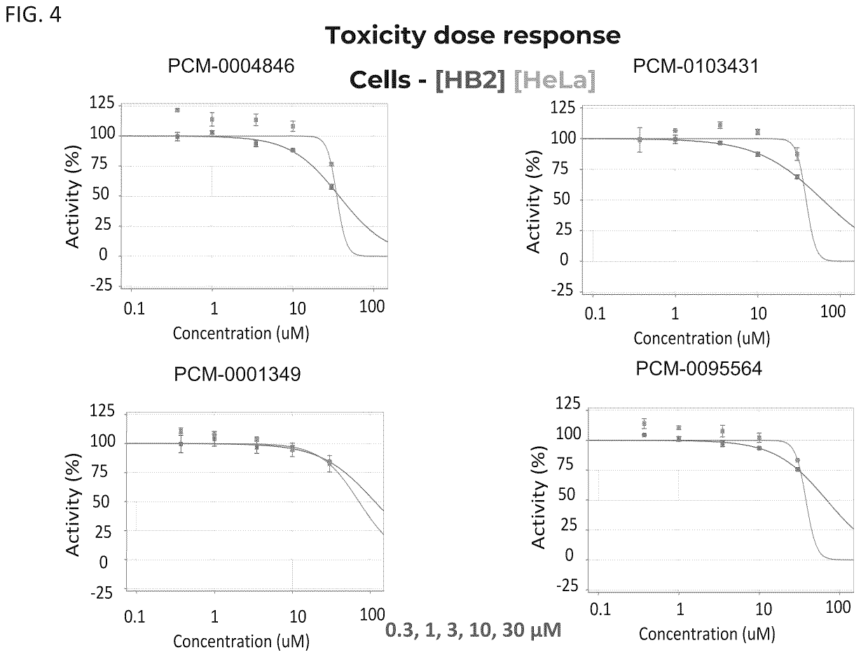

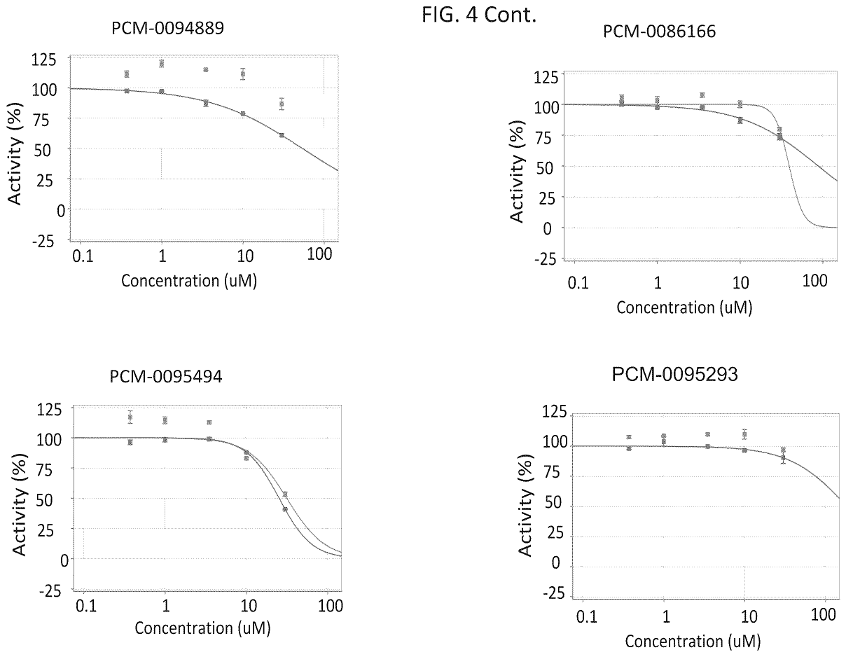

To examine the potential toxicity of the identified hits to host cells, the CellTiter-Glo (GTG) Luminescent Cell Viability Assay (G7570, Promega) was used (FIG. 4). Sterile tissue culture-treated white 384 flat bottom well plates (Greiner, #781080) were pre-plated with compounds at a final concentration of 0.3, 1, 3, 10, 30 μM using Echo555 acoustic transfer system (Labcyte, Germany). 8×10³ HeLa or HB2 epithelial cells were added to each well and incubated for 5 h. Gentamycin at a final concentration of 20 μg/ml was added to each well and the cells were incubated for overnight. CTG assay was performed in accordance to the manufacture's protocol. Shortly, medium was aspirated and 10 μl, of the CTG reagent was added to each well. Then the plates were shaken for 2 min and after 10 min incubation luminescent signal was detected using PheraStar FS plate reader (BMG Labtech, Ortenberg, Germany). The charts show the mean and standard error of the mean (SEM) of 2 biological repeats.

### Results

FIG. 4 shows that all eight compounds are not cytotoxic to HeLa nor to HB2 cells at 10 μM concentration.

### Example 5

### The Inhibitory Activity of Identified Compounds in a Dose-Response Assay

To test the inhibitory activity of identified compounds in a dose dependent manner, 4.6×10⁴ HeLa cells were seeded in each well of a sterile 96-well, cell culture-treated, flat-bottom white microplate and incubated at 37 °C 5% CO₂ humidified incubator for 4 h to let the cells adhere in the presence of 0, 0.25, 1, 3.25, 10, and 30 μM of compounds PCM-0094889, PCM-0086166, PCM-0004846, PCM-0103431, PCM-0095494, PCM-0095293, PCM-0001349, and PCM-0095564. Simultaneously, an overnight (12-16 h) S. Typhimurium culture expressing ssek3::lux was subcultured 1:100 in LB medium for 3 h at 37 °C on a shaker incubator to the late logarithmic phase (OD₆₀₀~1). Salmonella culture was then diluted 1:50 in DMEM and aliquots of 100 μl were added to each well to reach an MOI of 1:30 (cells per bacteria) and incubated at 37 °C. 5% CO₂ to allow infection. One h post-infection, gentamicin was added to a final concertation of 20 μg/ml and the plates were incubated for overnight. In the next day, the wells were washed three times with sterile PBS and bioluminescence was read by Infinite M-Plex Tecan plate reader. The charts show the mean and standard error of the mean (SEM) of three biological repeats in three independent experiments.

### Results

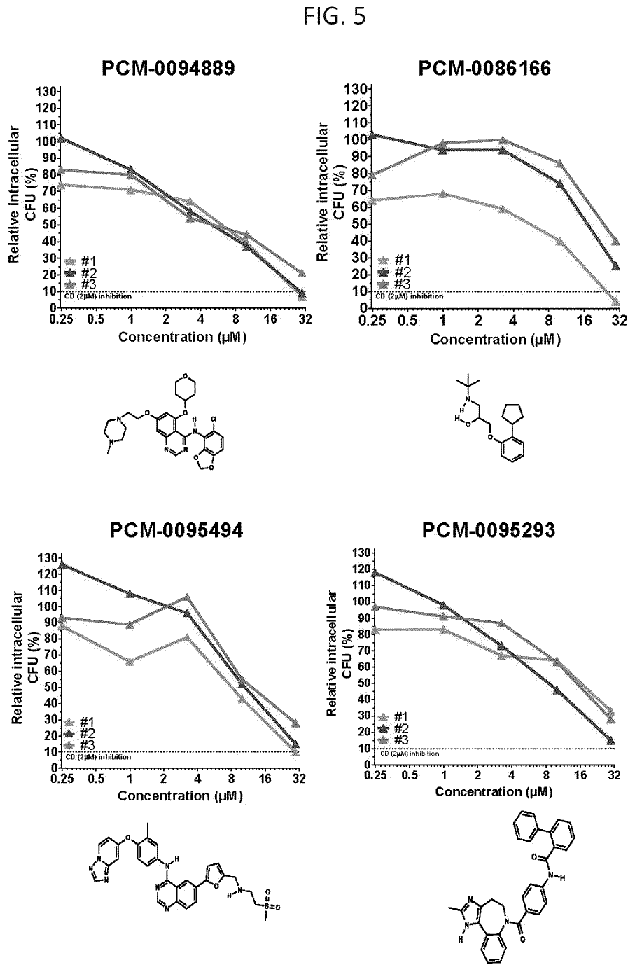

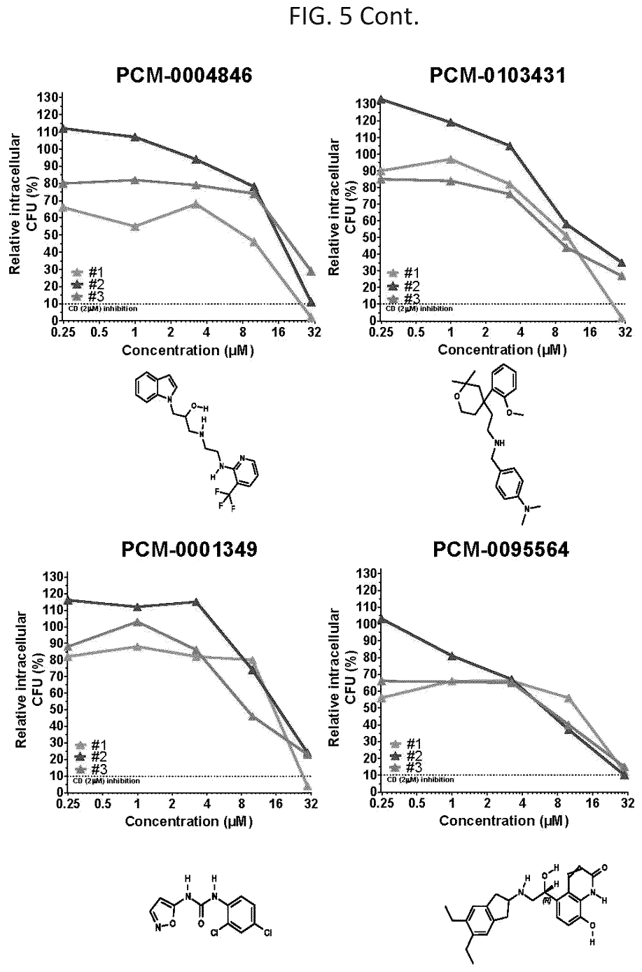

FIG. 5 shows the inhibitory activity of the eight compounds in a dose-dependent manner and its ability to reduce host cell infection by S. Typhimurium.

### Example 6

### The Identified Compounds Inhibit Salmonella Intracellular Replication Rather than Invasion

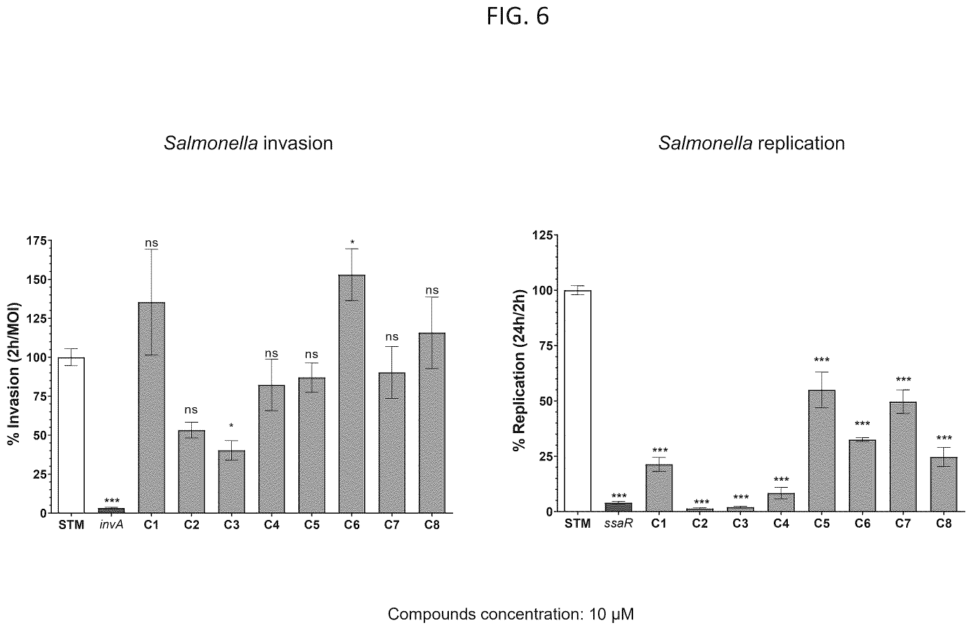

Reducing pathogen infection can be mediated either by inhibiting its invasion into host cells or by limiting its intracellular survival/replication. To distinguish between these options 5×10⁴ HeLa cells were seeded in 24-well (Greiner, #662160), cell culture-treated, flat-bottom microplate and incubated at 37 °C in a humidified 5% CO₂ incubator for 24 h prior to infection. In the next day, the medium was replaced with fresh DMEM medium containing the PCM-0094889, PCM-0086166, PCM-0004846, PCM-0103431, PCM-0095494, PCM-0095293, PCM-0001349, and PCM-0095564 compounds at a final concentration of 10 μM, which were incubated with the cells for 3 h. An overnight S. Typhimurium SL1344 culture was subcultured 1:100 into fresh LB medium and grown at 37 °C on a shaker incubator to the late logarithmic phase. After 3 h, the bacteria were diluted 1:100 in DMEM and 500 μl from the diluted bacteria were added into each well at MOI~1:50. The infected cells were spun down for 5 min at 1000 g and then incubated at 37 °C 5% CO₂. After 10 min, cells were washed three times with PBS and fresh medium supplemented with 10 μM of each compound were added to each well and further incubated for 20 min. At 30 min post infection, the medium was replaced with a fresh medium containing 100 μg/ml gentamicin and 10 μM of each compound and incubated for 90 min. At 2 h post infection, cells were washed three times with PBS and lysed with 250 μl lysis buffer (0.1% SDS, 1% Triton X-100 in PBS) by incubating the cells for 10 min at room temperature with gentle agitation. To calculate the number of invading bacteria at 2 h post infection (FIG. 6 left panel), the cell lysates were serially diluted in PBS and plated onto LB agar plates for CFU count. S. Typhimurium invasion in the presence of the inhibitors was calculated relative to S. Typhimurium (STM) invasion in the absence of the compounds. S. Typhimurium invA null mutant that is impaired in host cells invasion was included as a positive control.

To calculate the intracellular fold replication (FIG. 6 right panel), at 2 h post infection, the medium of the infected cells was replaced with DMEM supplemented 10 μg/ml gentamicin and 10 μM of each compound and incubated at 37 °C in a humidified 5% CO₂ incubator for. At 24 h post infection, the cells were washed three times with PBS and the lysed with 250 μl lysis buffer as above. Intracellular replication was determined by the ratio between the number of intracellular cells counted at 24 h post infection and their number at 2 h post infection.

S. Typhimurium replication in the presence of the inhibitors was calculated relative to its replication in the absence of the compounds. S. Typhimurium ssaR null mutant, which is impaired in host cells replication was included as a positive control. The charts show the mean and standard error of the mean (SEM) of seven biological repeats in two independent experiments.

### Results

FIG. 6 shows that all eight compounds can inhibit Salmonella intracellular replication rather than invasion and that this activity of the compounds limits the ability of the pathogen to infect and thrive within host cells.

### Example 7

### Identified Compounds were Affective in Inhibiting Intracellular Growth of S. Typhimurium in Bone Marrow Derived Macrophages (BMDMs)

Bone marrow-derived macrophage (BMDMs) were isolated from the femur leg bone of 7-week old SWISS female mice. Macrophages were diluted in BMDM medium [50% DMEM high glucose, 20% FBS, 30% L-929 conditioned medium, 2 mM L-glutamine, 1 mM sodium pyruvate, 50 nM β-mercaptoethanol] and seeded at 2.5×10⁵ cells/ml in a 24-well, cell culture-treated dish, 24 h prior to bacterial infection. In the next day, BMDM medium was replaced by BMDM fresh medium supplemented with 10 μM of the compounds (PCM-0094889, PCM-0086166, PCM-0004846, PCM-0103431, PCM-0095494, PCM-0095293, PCM-0001349, and PCM-0095564) and incubated for 3 h at 37 °C under 5% CO₂ atmosphere. Macrophages were infected at an MOI of 1:10 with cultures of S. Typhimurium SL1344 and its ΔssaR isogenic mutant that is impaired in intracellular replication as a negative control. Infected cells were spun down at 1000 g for 5 min and incubated for 30 min at 37 °C, 5% CO₂. Infection experiments were carried out using the gentamicin protection assay. At 30 min p.i, cells were washed three times with PBS to remove extracellular bacteria, and medium containing 100 μg/ml gentamicin was added for 1 h incubation. Wells were then washed three times with PBS and medium was replaced with fresh BMDM containing 10 μg/ml gentamicin. To determine intracellular growth of Salmonella at 2 and 20 h p.i., cells were washed three times with PBS and lysed with 250 μl of lysis buffer (0.1% SDS, 1% Triton X-100 in PBS). The number of CFUs in each well was quantified by plating serial dilutions of cell lysates on selective LB-agar plats. Salmonella uptake (left panel) was calculated as the number of intracellular bacteria recovered at 2 h p.i., divided by the infecting inoculum (CFUs). Salmonella survival (right panel) was calculated as the number of intracellular bacteria recovered at 24 h p.i., divided by intracellular bacteria recovered at 2 h p.i. The charts show the mean and standard error of the mean (SEM) of three biological repeats.

### Results

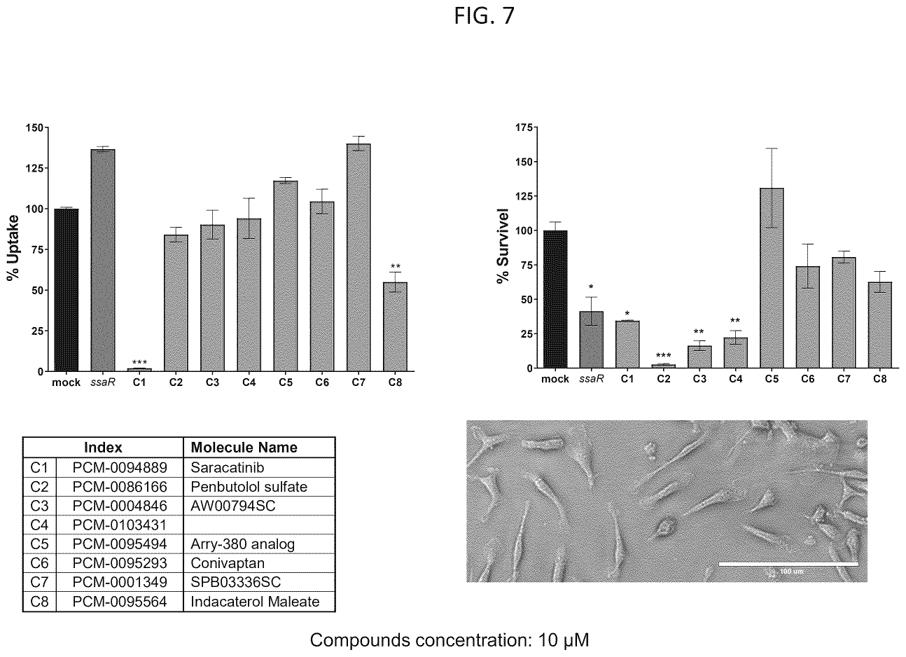

FIG. 7 shows that compounds 1~4 can effectively inhibit S. Typhimurium replication in mouse BMDM by more than 50%.

### Example 8

### The Identified Compounds can Inhibit the Intracellular Growth of Listeria monocytogenes

4×10⁵ HeLa cells were seeded in a 24-well tissue culture treated plate and incubated for 24 h at 37 °C under 5% CO₂ atmosphere. On the next day, cells were incubated for 3 h with fresh DMEM supplemented with the PCM-0094889, PCM-0086166, PCM-0004846, PCM-0103431, PCM-0095494, PCM-0095293, PCM-0001349, and PCM-0095564 compounds at a final concentration of 10 μM. overnight cultures of Listeria monocytogenes that were grown statically at 37 °C were washed and resuspended in PBS. Cell infection at am MOI of 1:50 was conducted by adding 20 μl of Listeria suspension to each well, following by incubation at 37 °C under 5% CO₂ atmosphere. 30 min p.i. the cells were washed with PBS and the medium was replaced with fresh medium supplemented with 10 μM of the tested compounds. One h p.i., gentamycin was added to each well at a final concentration of 50 μg/ml. At 6 h p.i., the cells were washed with PBS and lysed with 250 μl DDW at room temperature. The cell lysates were then diluted in PBS and plated on LB plates for CFU count. Intracellular Listeria infection was calculated as the number of intracellular bacteria recovered at 6 h p.i., divided by the infecting inoculum (CFUs). Listeria infection in the presence of the compounds is shown relative to the number of intracellular bacteria that were recovered from cells that were not treated with the compounds. The charts show the mean and standard error of the mean (SEM) of six biological repeats in two independent experiments, biological repeats.

### Results

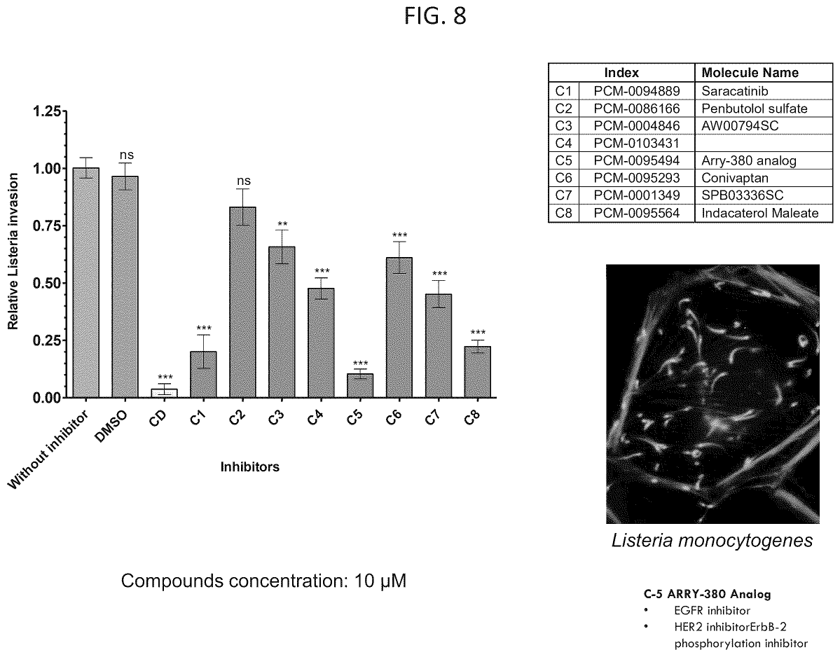

FIG. 8 shows that compounds 1, 4, 5, 7, and 8 were able to actively inhibit Listeria monocytogenes infection of HeLa cells. The fact that L. monocytogenes and S. Typhimurium are Gram-positive and Gram-negative bacteria, suggests that these compounds are active against very different bacteria and have the potential to inhibit infection by a wide range of pathogens, most likely via a host-dependent mechanism.

### Example 9

### The Identified Compounds Trigger Programmed Cell Death (PCD)

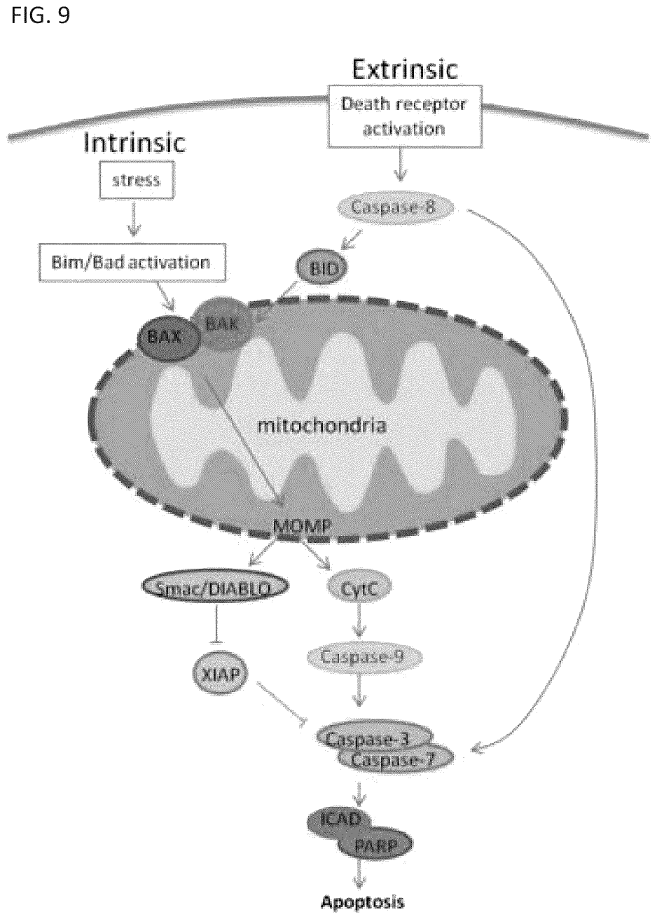

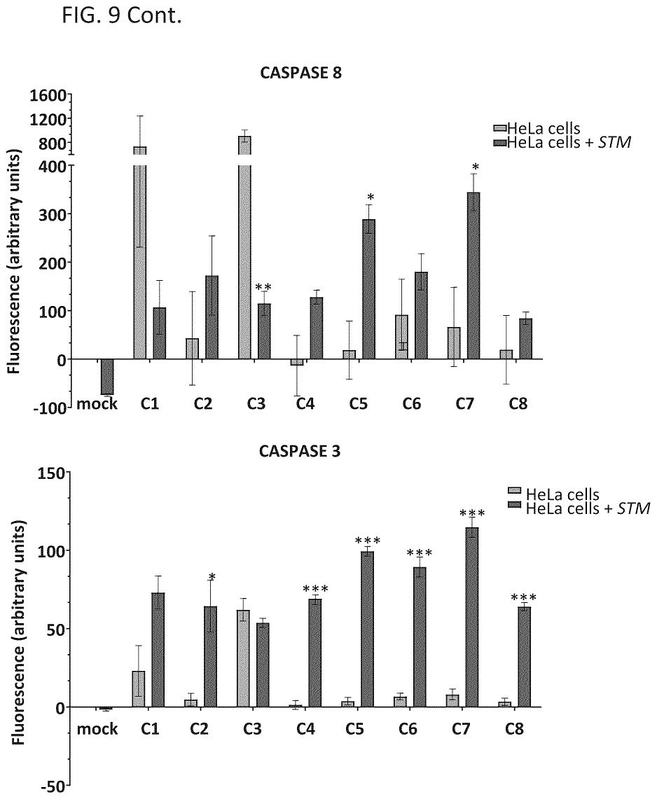

One of the possible mechanisms that could explain the activity of the compounds in inhibiting infection by different pathogens is inducing programed cell death in infected cells. To examine this experimentally, the present inventors have used Caspase-3, Caspase-8 and Caspase-9 Multiplex Activity Fluorometric Assay Kit (ab219915) to monitor caspase 3, caspase 8 and caspase 9 activity in HeLa cells infected with S. Typhimurium in the presence and absence of the compounds. The present inventors have found significantly higher activity of Caspase 8 in infected HeLa cells in the presence of compounds 5 and 7 and significantly higher activity of caspase 3 in infected cells when compounds 2, 4, 5, 6, 7, and 8 were present (see FIG. 9). These results suggest that these compounds enhance PCD by activating caspases 3 and 8 in infected cells, but not in uninfected cells and by that limit the replication of the pathogen in the host.

### Example 10

### Structure Activity Relationship (SAR) of Compounds PCM-0103431 and PCM-0001349

4.6×10⁴ HeLa cells were seeded in each well of a 96-well, cell culture-treated, flat-bottom white microplate (Greiner, #665083) and incubated at 37 °C. 5% CO₂ humidified incubator for 4 h to let the cells adhere, in the presence of 10 μM of compounds PCM-0103431 and PCM-0001349 and their chemical derivatives. As a positive control 2 μM Cytochalasin D (CD) was also included. Cells were infected with an overnight cultures of S. Typhimurium culture expressing the reporter system ssek3::lux, which was subcultured 1:100 in LB medium for 3 h at 37 °C and grown to the late logarithmic phase (OD₆₀₀~1). Salmonella culture was then diluted 1:50 in DMEM and aliquots of 100 μl were added to each well to reach an MOI of 1:50 (cells per bacteria) and incubated at 37 °C, 5% CO₂ to allow infection. One h p.i, gentamicin was added to a final concertation of 20 μg/ml and the infected cells were incubated for overnight. In the next day, the wells were washed three times with PBS and bioluminescence was read by Infinite M-Plex Tecan plate reader. FIG. 10A shows that chemical derivatives of compounds 4 and 7 can improve the inhibitory function of these compounds. Intracellular infection is shown relative to the Salmonella infection of HeLa cells in the presence of 0.1% DMSO. The charts show the mean and SEM of three biological repeats in three independent experiment. Chemicals derivatives of compounds PCM-0103431 and PCM-0001349 that presented enhanced inhibitory activity are highlighted by a red box, and their chemical structures are shown in FIG. 10B.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited in the Application

ADDIN EN.REFLIST 1. Chandra P, Rajmani R S, Verma G, Bhavesh N S, Kumar D. Targeting Drug-Sensitive and -Resistant Strains of Mycobacterium tuberculosis by Inhibition of Src Family Kinases Lowers Disease Burden and Pathology. mSphere 2016; 1.
2. J ON. Review on Antimicrobial Resistance. Antimicrobial Resistance: Tackling a Crisis for the Health and Wealth of Nations. 2014.
3. Kamaruzzaman N F, Kendall S, Good L. Targeting the hard to reach: challenges and novel strategies in the treatment of intracellular bacterial infections. Br J Pharmacol 2017; 174:2225-36.
4. Li L, Dickinson M S, Coers J, Miao E A. Pyroptosis in defense against intracellular bacteria. Semin Immunol 2023; 69:101805.
5. Petit T J P, Lebreton A. Adaptations of intracellular bacteria to vacuolar or cytosolic niches. Trends Microbiol 2022; 30:736-48.
6. Johnson R, Mylona E, Frankel G. Typhoidal Salmonella: Distinctive virulence factors and pathogenesis. Cell Microbiol 2018; 20: e12939.
7. Gal-Mor O. Persistent Infection and Long-Term Carriage of Typhoidal and Nontyphoidal Salmonellae. Clin Microbiol Rev 2019; 32.
8. Green D R. The Coming Decade of Cell Death Research: Five Riddles. Cell 2019; 177:1094-107.
9. Mogensen T H. Pathogen recognition and inflammatory signaling in innate immune defenses. Clin Microbiol Rev 2009; 22:240-73, Table of Contents.
10. Alara J A, Alara O R. An Overview of the Global Alarming Increase of Multiple Drug Resistant: A Major Challenge in Clinical Diagnosis. Infect Disord Drug Targets 2023.
11. Kellermann M, Scharte F, Hensel M. Manipulation of Host Cell Organelles by Intracellular Pathogens. Int J Mol Sci 2021; 22.
12. Liu Y, Jia Y, Yang K, Wang Z. Heterogeneous Strategies to Eliminate Intracellular Bacterial Pathogens. Front Microbiol 2020; 11:563.

What is claimed is:

1. A method of treating or preventing an infection by an intracellular pathogen in a subject in need thereof by administering to the subject a therapeutically effective amount of a pharmaceutical composition, or of inhibiting growth of an intracellular pathogen or invasion thereof into host cells by contacting the pathogen with a therapeutically effective amount of a pharmaceutical composition, wherein the pharmaceutical composition comprises at least one compound represented by Formula I, II, IV, V, VI or VII: 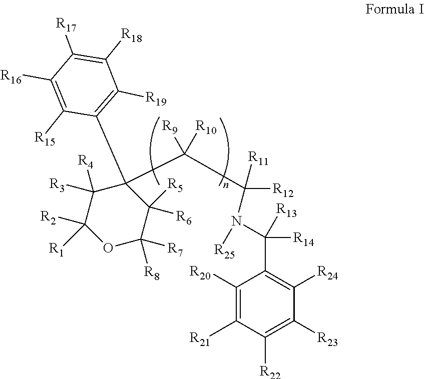
wherein:
n is 0 or 1;
R₁ and R₂ are each independently selected from hydrogen and alkyl, preferably a lower alkyl of 1 to 4 carbon atoms in length, wherein at least one of R₁ and R₂ is said alkyl;
R₃-R₁₄ are each independently selected from hydrogen, alkyl, cycloalkyl, halo, haloalkyl, hydroxy, alkoxy, thiol, thioalkoxy, amine, cyano and nitro;
R₁₅-R₂₄ are each independently selected from hydrogen, alkyl, cycloalkyl, amine, halo, hydroxy, thiol, alkoxy, and thioalkoxy; and
R₂₅ is selected from hydrogen, alkyl and cycloalkyl, 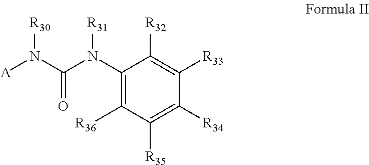
wherein:
R₃₀ and R₃₁ are each independently selected from hydrogen, alkyl and cycloalkyl;
R₃₂-R₃₆ are each independently selected from hydrogen, alkyl, cycloalkyl, halo, haloalkyl, hydroxy, alkoxy, thiol, thioalkoxy, amine, cyano and nitro, provided that at least one of R₃₂-R₃₆, preferably at least one of R₃₂, R₃₄ and R₃₆ is halo; and
A is selected from: 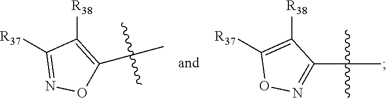
wherein the curved line represents an attachment point to NR₃₀₋; and R₃₇ and R₃₈ are each independently selected from hydrogen alkyl and cycloalkyl, 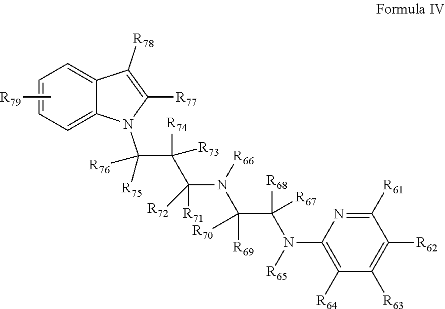
wherein:
R₆₁-R₆₄ are each independently selected from hydrogen, alkyl, cycloalkyl, halo, haloalkyl, amine, hydroxy, alkoxy, thiol, thioalkoxy;
R₆₅ and R₆₆ are each independently selected from hydrogen, alkyl and cycloalkyl;
R₆₇-R₇₈ are each independently selected from hydrogen, alkyl, cycloalkyl, halo, haloalkyl, amine, hydroxy, alkoxy, thiol and thioalkoxy; and
R₈₀ represents one or more (1, 2, 3, 4 or 5) substituents on the phenyl ring, or is absent, wherein each of these substituents, if present, is independently selected from alkyl, cycloalkyl, halo, haloalkyl, amine, hydroxy, alkoxy, thiol, and thioalkoxy, 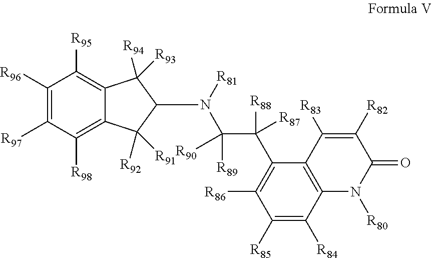
wherein:
R₈₀ and R₈₁ are each independently selected from hydrogen, alkyl and cycloalkyl;
R₈₂-R₈₆ are each independently selected from hydrogen, alkyl, cycloalkyl, halo, haloalkyl, amine, hydroxy, alkoxy, thiol, and thioalkoxy;
R₈₇-R₉₀ are each independently selected from hydrogen, alkyl, cycloalkyl, halo, haloalkyl, amine, hydroxy, alkoxy, thiol, and thioalkoxy;
R₉₁-R₉₄ are each independently selected from hydrogen, alkyl, cycloalkyl, halo, haloalkyl, amine, hydroxy, alkoxy, thiol, and thioalkoxy; and
R₉₅-R₉₈ are each independently selected from hydrogen, alkyl, cycloalkyl, halo, haloalkyl, amine, hydroxy, alkoxy, thiol, and thioalkoxy, 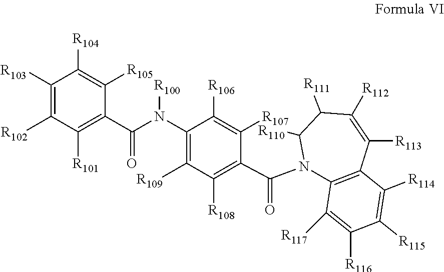
wherein:
R₁₀₀ is hydrogen, alkyl or cycloalkyl;
R₁₀₁-R₁₀₅ are each independently selected from hydrogen, alkyl, cycloalkyl, aryl, halo, haloalkyl, amine, hydroxy, alkoxy, thiol, thioalkoxy, heteroaryl, cyano, and nitro, wherein at least one of R₁₀₁-R₁₀₅, preferably R₁₀₁, is aryl;
R₁₀₆-R₁₀₉ are each independently selected from hydrogen, alkyl, cycloalkyl, halo, haloalkyl, amine, hydroxy, alkoxy, thiol and thioalkoxy;
R₁₁₀-R₁₁₃ are each independently selected from hydrogen, alkyl, cycloalkyl, halo, haloalkyl, amine, hydroxy, alkoxy, thiol and thioalkoxy, or, alternatively, two of R₁₁₀-R₁₁₃ form together a substituted or unsubstituted cyclic ring (alicyclic, heteroalicyclic, aryl or heteroaryl); and
R₁₁₄-R₁₁₇ are each independently selected from hydrogen, alkyl, cycloalkyl, halo, haloalkyl, amine, hydroxy, alkoxy, thiol and thioalkoxy, 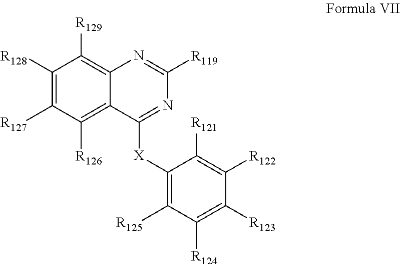
wherein:
X is O, S or NR₁₂₀;
R₁₁₉ and R₁₂₀ (if present) are each independently selected from hydrogen, alkyl or cycloalkyl;
R₁₂₁-R₁₂₅ are each independently selected from hydrogen, alkyl, cycloalkyl, aryl, halo, haloalkyl, amine, hydroxy, alkoxy, aryloxy, thioaryloxy, thiol, thioalkoxy, heteroaryl, cyano and nitro, or, alternatively or in addition, two of R₁₂₁-R₁₂₅ form together a substituted or unsubstituted cyclic ring (alicyclic, heteroalicyclic, aryl or heteroaryl); and
R₁₂₆-R₁₂₉ are each independently selected from hydrogen, alkyl, cycloalkyl, halo, haloalkyl, amine, hydroxy, alkoxy, thiol, thioalkoxy, heteroalicyclic, heteroaryl, and aryloxy,
provided that at least one of R₁₂₁-R₁₂₅ and at least one of R₁₂₆-R₁₂₉ is other than hydrogen.

2. The method of claim 1, wherein said intracellular pathogen is selected from the group consisting of bacterial pathogen, viral pathogen, fungal pathogen and a protozoan pathogen.

3. The method of claim 1, wherein said intracellular pathogen comprises bacteria.

4. The method of claim 1, wherein said bacteria is Gram positive or Gram negative; or is of a genus selected from the group consisting of Listeria monocytogenes, Salmonella enterica serovars (e.g., Salmonella Typhi), Mycobacterium tuberculosis, Chlamydia trachomatis, Rickettsia species and Legionella pneumophila; or comprises a vacuolar intracellular bacterium; or comprises an intracellular cytosolic bacterium.

5. The method of claim 1, wherein said compound is represented by said Formula I.

6. The method of claim 1, wherein:
R₁ and R₂ are each independently selected from methyl, ethyl, propyl and isopropyl; and/or
each of R₁₅-R₁₉ is hydrogen, or at least one of R₁₅-R₁₉ is selected from alkyl, fluoro, and alkoxy; and/or
at least one of R₂₀-R₂₄ is selected from amine, fluoro, and alkoxy.

7. The method of claim 1, wherein said compound is represented by said Formula II.

8. The method of claim 7, wherein:
A is 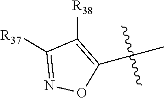
and at least two of R₃₂-R₃₆, preferably at least two of R₃₂, R₃₄ and R₃₆, are each halo, preferably chloro; or
A is 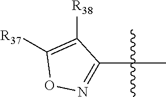
and no more than one of R₃₂-R₃₆, preferably one of R₃₂, R₃₄ and R₃₆, is halo, preferably chloro.

9. The method of claim 1, wherein said compound is represented by Formula IV.

10. The method of claim 9, wherein:
at least one of R₆₁-R₆₄, preferably R₆₄, is selected from alkyl, haloalkyl and halo, and is preferably a trihaloalkyl; and/or
at least one of R₇₁-R₇₆, preferably R₇₄, is hydroxy.

11. The method of claim 1, wherein said compound is represented by Formula V.

12. The method of claim 11, wherein:
at least one of R₈₄-R₈₆, preferably R₈₄, is hydroxy; and/or
at least one of R₈₇-R₉₀, preferably R₈₇, is hydroxy; and/or
at least one, or at least two of R₉₅-R₉₈ is an alkyl.

13. The method of claim 1, wherein said compound is represented by Formula VI.

14. The method of claim 13, wherein at least one of R₁₀₁-R₁₀₅ is aryl; and/or at least two of R₁₁₀-R₁₁₃ form together a substituted or unsubstituted cyclic ring, preferably a substituted or unsubstituted heteroaryl.

15. The method of claim 1, wherein said compound is represented by Formula VII.

16. The method of claim 15, wherein the compound is represented by Formula VIIa: 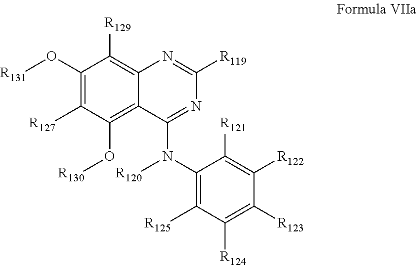
wherein:
R₁₂₁ and R₁₂₂ form together said cyclic ring, preferably said heteroalicyclic ring; and
R₁₃₀ and R₁₃₁ are each independently selected from alkyl, cycloalkyl, and heteroalicyclic.

17. The method of claim 16, wherein:
R₁₃₀ and R₁₃₁ each independently is or comprises a heteroalicyclic; and/or
R₁₃₁ is an alkyl substituted by a heteroalicylic, preferably a piperazine; and/or
at least one of R₁₂₁-R₁₂₅ is aryloxy, preferably heteroaryloxy; and/or
at least one of R₁₂₆-R₁₂₉ is a heteroaryl, preferably furyl, more preferably a substituted furyl.

18. The method of claim 15, wherein R₁₂₇ is said heteroaryl and the compound is represented by Formula VIIb: 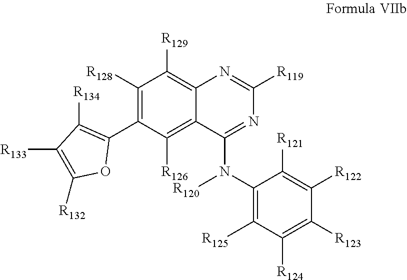
wherein:
at least one of R₁₂₁-R₁₂₅ is said aryloxy, preferably a heteroaryloxy; and
R₁₃₂-R₁₃₄ are each independently selected from hydrogen, alkyl, cycloalkyl, halo, haloalkyl, amine, heteroalicyclic, heteroaryl, cyano and nitro.

19. The method of claim 18, wherein R₁₂₃ is heteroaryloxy, preferably 7-triazolopyridinoxy; and/or at least one of R₁₃₂-R₁₃₄, preferably R₁₃₂, is an alkyl, preferably a substituted alkyl.

20. A method of treating or preventing an infection by an intracellular pathogen in a subject in need thereof by administering to the subject a therapeutically effective amount of a pharmaceutical composition, or of inhibiting growth of an intracellular pathogen or invasion thereof into host cells by contacting the pathogen with a therapeutically effective amount of a pharmaceutical composition, wherein the pharmaceutical composition comprises a compound which induces programmed cell death (PCD) in host cells of said intracellular pathogen, thereby treating or preventing the infection.
